(19) United States
(12) Patent Application Publication
(10) Pub. No.: US 2025/0296966 A1
(43) Pub. Date: Sep. 25, 2025

(54) COMBINATION OF AN ANTIBODY SPECIFIC FOR A TUMOR ANTIGEN AND A CD47 INHIBITOR

(71) Applicants: DAIICHI SANKYO COMPANY, LIMITED, Tokyo (JP); AstraZeneca UK Limited, Cambridge (GB)

(72) Inventors: Norihito KAWASAKI, Tokyo (JP); Saori ISHIDA, Tokyo (JP); Mayumi SUE, Tokyo (JP); Takafumi OTSUKA, Tokyo (JP)

(73) Assignees: DAIICHI SANKYO COMPANY, LIMITED, Tokyo (JP); AstraZeneca UK Limited, Cambridge (GB)

(21) Appl. No.: 18/863,639

(22) PCT Filed: May 10, 2023
(86) PCT No.: PCT/IB2023/054841

§ 371 Date: Nov. 7, 2024

Related U.S. Application Data
(60) Provisional application No. 63/340,745, filed on May 11, 2022.

(51) Int. Cl. C07K 14/47; A61K 38/00; A61K 47/68; A61P 35/00; C07K 16/28

(52) U.S. Cl. CPC C07K 14/4703; A61K 47/6811; A61K 47/6855; A61P 35/00; C07K 16/2803; A61K 38/00; C07K 2319/31

(57) ABSTRACT

The present invention relates to a CD47 inhibitor for use in the treatment or prevention of cancer by simultaneous or sequential administration with an antibody specific for a tumor antigen. The invention also relates to a pharmaceutical composition comprising: a CD47 inhibitor; and an antibody specific for a tumor antigen.

### TECHNICAL FIELD

The present invention relates to a pharmaceutical composition comprising a CD47 inhibitor. The present invention also relates to a CD47 inhibitor for use in the treatment or prevention of cancer and also a therapeutic method comprising administering a CD47 inhibitor.

### BACKGROUND ART

CD47 (also known as integrin-associated protein-IAP) is a 50 kDa transmembrane protein receptor that has an extracellular N-terminal IgV domain, five transmembrane domains and a short C-terminal intracellular tail. CD47 is expressed on red blood cells as a marker of “self” and is also highly expressed on tumor cells. It has been observed that high CD47 expression on tumor cells can act, in acute myeloid leukaemia and several solid tumor cancers, as a negative prognostic factor for survival (Patent Literature 1).

SIRPα (SHPS-1) is a single transmembrane molecule belonging to the Ig superfamily present in myeloid cells such as macrophages, dendritic cells and neutrophils, and glial cells (Non-Patent Literature 1). The extracellular region thereof consists of a single IgV domain and two IgC domains. The IgV domain (also referred to as the “D1 domain” (Non-Patent Literature 2)), which is a connecting position to CD47, is reported to have 10 variants in humans (Non-Patent Literature 3). On the other hand, the intracellular region thereof contains immunoreceptor tyrosine-based inhibition motifs (ITIM). Upon the extracellular region of SIRPα binds to CD47, the binding to tyrosine dephosphorylation enzymes, SHP-1 and SEPHP-2, is induced and a suppressive signal is transmitted.

It is reported that SIRPα-CD47 interaction causes a physiological phenomenon, i.e., CD47 on red blood cells binds to SIRPα on macrophages to transmit a “Don't eat me” signal, with the result that unwanted phagocytosis by red blood cells can be avoided (Non-Patent Literature 4). Also, under tumor microenvironments, it is suggested that when CD47, which as noted above is highly expressed on tumor cells, binds to SIRPα on macrophages and dendritic cells, phagocytic activity to engulf tumor cells is suppressed. When phagocytic activity is suppressed, the subsequent tumor antigen presentation to T cells and further subsequent tumor immune response is suppressed. Thus, an immune phenomenon, that is, phagocytosis of tumor cells, is considered as a checkpoint of entry of a tumor antigen.

Patent Literature 1 discloses a construct which binds to the CD47 protein for use in cancer therapy. The construct is a polypeptide which comprises an SIRPα D1 domain and an Fc domain monomer linked to the N-terminus or the C-terminus thereof.

An antibody-drug conjugate (ADC) comprises a drug with cytotoxicity conjugated to an antibody, whose antigen is expressed on the surface of cancer cells and which also binds to an antigen capable of cellular internalization. ADCs can therefore deliver the drug selectively to cancer cells in order to cause accumulation of the drug within cancer cells and to kill the cancer cells.

As one such antibody-drug conjugate, an antibody-drug conjugate comprising an antibody and a derivative of exatecan, which is a topoisomerase I inhibitor, as its components are known (Patent Literature 2 to 8, Non-Patent Literature 5 to 8).

Patent Literature 2 to 8 disclose that an antibody-drug conjugate as mentioned above can be administered in combination with any one of various cancer therapeutic agents.

However, none of the test results show a superior combined effect when the foregoing antibody-drug conjugate is used in combination with CD47 inhibitor, nor has there been any disclosure of a scientific basis for suggesting such a result.

### CITATION LIST

### Patent Literature

[Patent Literature 1] International Publication No. WO 2017/027422
[Patent Literature 2] International Publication No. WO 2014/057687
[Patent Literature 3] International Publication No. WO 2014/061277
[Patent Literature 4] International Publication No. WO 2015/098099
[Patent Literature 5] International Publication No. WO 2015/115091
[Patent Literature 6 International Publication No. WO 2015/146132
[Patent Literature 7] International Publication No. WO 2015/155976
[Patent Literature 8] International Publication No. WO 2015/155998

### Non-Patent Literature

[Non-Patent Literature 1] Matozaki et al. Trends in cell biol. 2009 (19) 2, 72-80
[Non-Patent Literature 2] Lee W Y, J Immunol, 2007, 7741-7750
[Non-Patent Literature 3] Takenaka et al. Nat Immunol. 2007 (8) 12, 1313-1323
[Non-Patent Literature 4] Matozaki et al. J. Biochem. 2014 (155) 6, 335-344
[Non-Patent Literature 5] Ogitani Y. et al., Clinical Cancer Research (2016) 22 (20), 5097-5108.
[Non-Patent Literature 6] Ogitani Y. et al., Cancer Science (2016) 107, 1039-1046.
[Non-Patent Literature 7] Doi T, et al., Lancet Oncol 2017; 18:1512-22.
[Non-Patent Literature 8] Takegawa N, et al., Int. J. Cancer: 141, 1682-1689 (2017)

### SUMMARY OF INVENTION

### Technical Problem

Antibodies specific for tumor-associated antigens are known to have therapeutic effects in cancer patients. However, there is a need for improved therapies for treating cancers, either where such antibodies, alone, do not give rise to complete patient remission or in situations where it is desirable to reduce the necessary dosage of such antibodies in order to be therapeutic. Therefore, there is a need for obtaining a superior antitumor effect from such antibodies.

An object of the present invention is to provide a pharmaceutical composition wherein an inhibitor of the CD47 protein and an antibody specific for a tumor antigen are provided in combination, and/or a therapeutic method comprising administering an inhibitor of the CD47 protein and an antibody specific for a tumor antigen in combination to a subject.

### Solution to Problem

As a result of diligent studies in order to solve the above problems, the present inventors have found that combined administration of a CD47 inhibitor and an antibody specific for a tumor antigen exhibits a superior combined effect, and completed the present invention. Specifically, the present invention includes the following aspects of the invention.

(1) A pharmaceutical composition comprising:
i. a CD47 inhibitor; and
ii. an antibody specific for a tumor antigen.
   (2) A pharmaceutical composition according to (1), wherein the CD47 inhibitor comprises SIRPα or a SIRPα derivative.
   (3) A pharmaceutical composition according to (2), wherein the SIRPα derivative comprises a polypeptide having a sequence with at least 80%, 90%, 95%, 99% or 100% sequence identity to the residues 1 to 149 of SEQ ID NO: 15 or 16.
   (4) A pharmaceutical composition according to (1) or (2), wherein the CD47 inhibitor is a fusion protein and further comprises an Fc region.
   (5) A pharmaceutical composition according to (4), wherein the CD47 inhibitor comprises a polypeptide having a sequence with at least 80%, 90%, 95%, 99% or 100% sequence identity to SEQ ID NO: 15 or 16.
   (6) A pharmaceutical composition according to any one of (1) to (5), wherein the CD47 inhibitor is Evorpacept.
   (7) A pharmaceutical composition according to (2), wherein the CD47 inhibitor is selected from the group consisting of: TTI-621, TTI-622, DSP-107 and SL-172154.
   (8) A pharmaceutical composition according to (1) wherein the CD47 inhibitor comprises an antibody or an antigen-binding fragment thereof, wherein the antibody is specific for CD47.
   (9) A pharmaceutical composition according to (8), wherein the antibody specific for CD47 is selected from the group consisting of: Magrolimab, Lemsoparlimab, AO-176, SRF-231, IBI-188, IBI-322, IMC-002, MIL-95, TG-1801, ZL-1201, AK-117 (Ligufalimab) and IMM-0306.
   (10) A pharmaceutical composition according to (1), wherein the CD47 inhibitor comprises a small-molecule agent capable of binding to CD47.
   (11) A pharmaceutical composition according to (10), wherein the small molecule agent is selected from the group consisting of RRx-001 and IMM-01.
   (12) A pharmaceutical composition according to any one of (1) to (11), wherein CD47 comprises a polypeptide sequence represented by SEQ ID NO: 13.
   (13) A pharmaceutical composition according to any one of (1) to (12) further comprising an antibody-drug conjugate, wherein the antibody specific for a tumor antigen is a part of the antibody-drug conjugate, and wherein the antibody-drug conjugate further comprises a linker and a drug, the antibody specific for a tumor antigen being connected to the drug via the linker, the linker and the drug forming a drug-linker.
   (13a) A pharmaceutical composition according to any one of (1) to (12) further comprising an antibody-drug conjugate wherein the antibody-drug conjugate comprises the antibody specific for a tumor antigen connected via a linker to a drug, the linker and the drug forming a drug-linker.
   (14) A pharmaceutical composition according to (13) or (13a), wherein the drug-linker is as represented by the following formula:

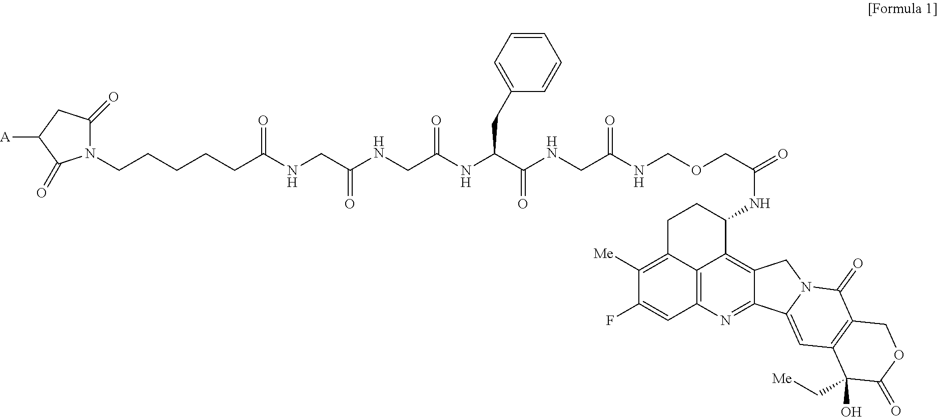

wherein A represents the connecting position to the antibody, and wherein the drug-linker is conjugated to the antibody specific for a tumor antigen via a thioether bond.
(15) A pharmaceutical composition according to any one of (1) to (14), wherein the antibody specific for a tumor antigen is an anti-HER2 antibody, an anti-HER3 antibody, an anti-TROP2 antibody, an anti-B7-H3 antibody, an anti-GPR20 antibody, or an anti-CDH6 antibody.
(16) A pharmaceutical composition according to (15), wherein the antibody specific for a tumor antigen is the anti-HER2 antibody.
(17) A pharmaceutical composition according to (16), wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.
(18) A pharmaceutical composition according to (16), wherein the anti-HER2 antibody is an antibody comprising a heavy chain consisting of an amino acid sequence represented by SEQ ID NO: 1 and a light chain consisting of an amino acid sequence represented by SEQ ID NO: 2.
(19) A pharmaceutical composition according to any one of (16) to (18) as dependent on (14), wherein the average number of units of the drug-linker conjugated per antibody molecule in the antibody-drug conjugate is in the range of from 7 to 8.
(20) The pharmaceutical composition according to (15), wherein the antibody specific for a tumor antigen is an anti-HER3 antibody.
(21) The pharmaceutical composition according to (20), wherein the anti-HER3 antibody is an antibody comprising a heavy chain consisting of an amino acid sequence represented by SEQ ID NO: 3 and a light chain consisting of an amino acid sequence represented by SEQ ID NO: 4.
(22) The pharmaceutical composition according to (21), wherein the anti-HER3 antibody lacks a lysine residue at the carboxyl terminus of the heavy chain.
(23) The pharmaceutical composition according to any one of (20) to (22) as dependent on (14), wherein the average number of units of the drug-linker conjugated per antibody molecule in the antibody-drug conjugate is in the range of from 7 to 8.
(24) The pharmaceutical composition according to (15), wherein the antibody specific for a tumor antigen is an anti-TROP2 antibody.
(25) The pharmaceutical composition according to (24), wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.
(26) The pharmaceutical composition according to (25), wherein the anti-TROP2 antibody lacks a lysine residue at the carboxyl terminus of the heavy chain.
(27) The pharmaceutical composition according to any one of (24) to (26) as dependent on (14), wherein the average number of units of the drug-linker conjugated per antibody molecule in the antibody-drug conjugate is in the range of from 3.5 to 4.5.
(28) The pharmaceutical composition according to (15), wherein the antibody specific for a tumor antigen is an anti-B7-H3 antibody.
(29) The pharmaceutical composition according to (28), wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.
(30) The pharmaceutical composition according to (29), wherein the anti-B7-H3 antibody lacks a lysine residue at the carboxyl terminus of the heavy chain.
(31) The pharmaceutical composition according to any one of (28) to (30) as dependent upon (14), wherein the average number of units of the drug-linker conjugated per antibody molecule in the antibody-drug conjugate is in the range of from 3.5 to 4.5.
(32) The pharmaceutical composition according to (15), wherein the antibody specific for a tumor antigen is an anti-GPR20 antibody.
(33) The pharmaceutical composition according to (32), wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.
(34) The pharmaceutical composition according to (33), wherein the anti-GPR20 antibody lacks a lysine residue at the carboxyl terminus of the heavy chain.
(35) The pharmaceutical composition according to any one of (32) to (34) as dependent on (14), wherein the average number of units of the drug-linker conjugated per antibody molecule in the antibody-drug conjugate is in the range of from 7 to 8.
(36) The pharmaceutical composition according to (15), wherein the antibody specific for a tumor antigen is an anti-CDH6 antibody.
(37) The pharmaceutical composition according to (36), wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.
(38) The pharmaceutical composition according to (37), wherein the anti-CDH6 antibody lacks a lysine residue at the carboxyl terminus of the heavy chain.
(39) The pharmaceutical composition according to any one of (36) to (38) as dependent on (14), wherein the average number of units of the drug-linker conjugated per antibody molecule in the antibody-drug conjugate is in the range of from 7 to 8.
(40) A CD47 inhibitor for use in the treatment or prevention of cancer by simultaneous or sequential administration with an antibody specific for a tumor antigen.
(41) A CD47 inhibitor for use according to (40), wherein the CD47 inhibitor comprises SIRPα or a SIRPα derivative.
(42) A CD47 inhibitor for use according to (41), wherein the SIRPα derivative comprises a polypeptide having a sequence with at least 80%, 90%, 95%, 99% or 100% sequence identity to the residues 1 to 149 of SEQ ID NO: 15 or 16.
(43) A CD47 inhibitor for use according to claim 40) or (41), wherein the CD47 inhibitor is a fusion protein and further comprises an Fc region.
(44) A CD47 inhibitor for use according to (43), wherein the CD47 inhibitor comprises a polypeptide having a sequence with at least 80%, 90%, 95%, 99% or 100% sequence identity to SEQ ID NO: 15 or 16.
(45) A CD47 inhibitor for use according to any one of (40) to (44), wherein the CD47 inhibitor is Evorpacept.
(46) A CD47 inhibitor for use according to (41), wherein the CD47 inhibitor is selected from the group consisting of: TTI-621, TTI-622, DSP-107 and SL-172154.
(47) A CD47 inhibitor for use according to (40) wherein the CD47 inhibitor comprises an antibody or an antigen-binding fragment thereof, wherein the antibody is specific for CD47.
(48) A CD47 inhibitor for use according to (47), wherein the antibody specific for CD47 is selected from the group consisting of: Magrolimab, Lemsoparlimab, AO-176, SRF-231, IBI-188, IBI-322, IMC-002, MIL-95, TG-1801, ZL-1201, AK-117 (Ligufalimab) and IMM-0306.
(49) A CD47 inhibitor for use according to (40), wherein the CD47 inhibitor comprises a small-molecule agent capable of binding to CD47.
(50) A CD47 inhibitor for use according to (49), wherein the small molecule agent is selected from the group consisting of RRx-001 and IMM-01.
(51) A CD47 inhibitor for use according to any one of (40) to (50), wherein CD47 comprises a polypeptide sequence represented by SEQ ID NO: 13.
(52) A CD47 inhibitor for use according to any one of (40) to (51) wherein the antibody specific for a tumour antigen is part of an antibody-drug conjugate and wherein the antibody-drug conjugate comprises the antibody specific for a tumor antigen connected via a linker to a drug, the linker and the drug forming a drug-linker.
(53) A CD47 inhibitor for use according to (52), wherein the drug-linker is as represented by the following formula:

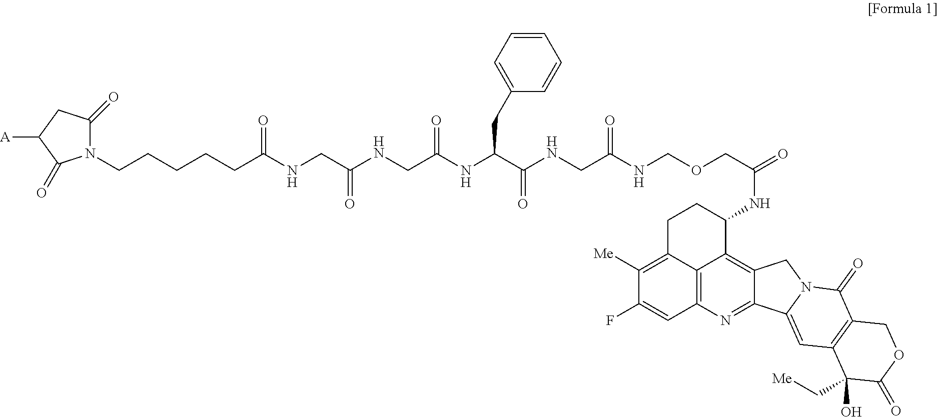

wherein A represents the connecting position to the antibody, and wherein the drug-linker is conjugated to the antibody specific for a tumor antigen via a thioether bond.
(54) A CD47 inhibitor for use according to any one of (40) to (53), wherein the antibody specific for a tumor antigen is an anti-HER2 antibody, an anti-HER3 antibody, an anti-TROP2 antibody, an anti-B7-H3 antibody, an anti-GPR20 antibody, or an anti-CDH6 antibody.
(55) A CD47 inhibitor for use according to (54), wherein the antibody specific for a tumor antigen is the anti-HER2 antibody.
(56) A CD47 inhibitor for use according to (55), wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.
(57) A CD47 inhibitor for use according to (55), wherein the anti-HER2 antibody is an antibody comprising a heavy chain consisting of an amino acid sequence represented by SEQ ID NO: 1 and a light chain consisting of an amino acid sequence represented by SEQ ID NO: 2.
(58) A CD47 inhibitor for use according to any one of (55) to (57) as dependent on (53), wherein the average number of units of the drug-linker conjugated per antibody molecule in the antibody-drug conjugate is in the range of from 7 to 8.
(59) A CD47 inhibitor for use according to (54), wherein the antibody specific for a tumor antigen is an anti-HER3 antibody.
(60) A CD47 inhibitor for use according to (59), wherein the anti-HER3 antibody is an antibody comprising a heavy chain consisting of an amino acid sequence represented by SEQ ID NO: 3 and a light chain consisting of an amino acid sequence represented by SEQ ID NO: 4.
(61) A CD47 inhibitor for use according to (60), wherein the anti-HER3 antibody lacks a lysine residue at the carboxyl terminus of the heavy chain.
(62) A CD47 inhibitor for use according to any one of (59) to (61) as dependent on (53), wherein the average number of units of the drug-linker conjugated per antibody molecule in the antibody-drug conjugate is in the range of from 7 to 8.
(63) A CD47 inhibitor for use according to (54), wherein the antibody specific for a tumor antigen is an anti-TROP2 antibody.
(64) A CD47 inhibitor for use according to (63), wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.
(65) A CD47 inhibitor for use according to (64), wherein the anti-TROP2 antibody lacks a lysine residue at the carboxyl terminus of the heavy chain.
(66) A CD47 inhibitor for use according to any one of (63) to (65) as dependent on (53), wherein the average number of units of the drug-linker conjugated per antibody molecule in the antibody-drug conjugate is in the range of from 3.5 to 4.5.
(67) A CD47 inhibitor for use according to (54), wherein the antibody specific for a tumor antigen is an anti-B7-H3 antibody.
(68) A CD47 inhibitor for use according to (67), wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.
(69) A CD47 inhibitor for use according to (68), wherein the anti-B7-H3 antibody lacks a lysine residue at the carboxyl terminus of the heavy chain.
(70) A CD47 inhibitor for use according to any one of (67) to (69) as dependent upon (53), wherein the average number of units of the drug-linker conjugated per antibody molecule in the antibody-drug conjugate is in the range of from 3.5 to 4.5.
(71) A CD47 inhibitor for use according to (54), wherein the antibody specific for a tumor antigen is an anti-GPR20 antibody.
(72) A CD47 inhibitor for use according to (71), wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.
(73) A CD47 inhibitor for use according to (72), wherein the anti-GPR20 antibody lacks a lysine residue at the carboxyl terminus of the heavy chain.
(74) A CD47 inhibitor for use according to any one of (71) to (73) as dependent on (53), wherein the average number of units of the drug-linker conjugated per antibody molecule in the antibody-drug conjugate is in the range of from 7 to 8.
(75) A CD47 inhibitor for use according to (54), wherein the antibody specific for a tumor antigen is an anti-CDH6 antibody.
(76) A CD47 inhibitor for use according to (75), wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.
(77) A CD47 inhibitor for use according to (76), wherein the anti-CDH6 antibody lacks a lysine residue at the carboxyl terminus of the heavy chain.
(78) A CD47 inhibitor for use according to any one of (75) to (77) as dependent on (53), wherein the average number of units of the drug-linker conjugated per antibody molecule in the antibody-drug conjugate is in the range of from 7 to 8.
(79) A CD47 inhibitor for use according to any one of (40) to (78) wherein the CD47 inhibitor and the antibody specific for a tumor antigen are separately contained active components in different formulations.
(80) A CD47 inhibitor for use according to any one of (40) to (79) wherein the cancer is at least one selected from the group consisting of: breast cancer, gastric cancer, colorectal cancer, lung cancer, esophageal cancer, salivary gland cancer, esophagogastric junction adenocarcinoma, biliary tract cancer, Paget's disease, pancreatic cancer, ovarian cancer, bladder cancer, prostate cancer, uterine cancer, sarcoma, head and neck cancer, hepatocellular cancer, cervical cancer, brain tumor, glioma, eye tumor, thyroid cancer, thymus cancer, gallbladder cancer, lymphoma, leukemia, and myelodysplastic syndrome.
(81) A therapeutic method comprising administering a CD47 inhibitor and an antibody specific for a tumor antigen in combination to a subject in need of treatment.
(82) A therapeutic method according to (81), wherein the CD47 inhibitor comprises SIRPα or a SIRPα derivative.
(83) A therapeutic method according to (82), wherein the SIRPα derivative comprises a polypeptide having a sequence with at least 80%, 90%, 95%, 99% or 100% sequence identity to the residues 1 to 149 of SEQ ID NO: 15 or 16.
(84) A therapeutic method according to (81) or (82), wherein the CD47 inhibitor is a fusion protein and further comprises an Fc region.
(85) A therapeutic method according to (84), wherein the CD47 inhibitor comprises a polypeptide having a sequence with at least 80%, 90%, 95%, 99% or 100% sequence identity to SEQ ID NO: 15 or 16.
(86) A therapeutic method according to any one of (81) to (85), wherein the CD47 inhibitor is Evorpacept.
(87) A therapeutic method according to (82), wherein the CD47 inhibitor is selected from the group consisting of: TTI-621, TTI-622, DSP-107 and SL-172154.
(88) A therapeutic method according to (81) wherein the CD47 inhibitor comprises an antibody antigen-binding fragment thereof, wherein the antibody is specific for CD47.
(89) A therapeutic method according to (88), wherein the antibody specific for CD47 is selected from the group consisting of: Magrolimab, Lemsoparlimab, AO-176, SRF-231, IBI-188, IBI-322, IMC-002, MIL-95, TG-1801, ZL-1201, AK-117 (Ligufalimab) and IMM-0306.
(90) A therapeutic method according to (81), wherein the CD47 inhibitor comprises a small-molecule agent capable of binding to CD47.
(91) A therapeutic method according to (90), wherein the small molecule agent is selected from the group consisting of RRx-001 and IMM-01.
(92) A therapeutic method according to any one of (81) to (91), wherein CD47 comprises a polypeptide sequence represented by SEQ ID NO: 13.
(93) A therapeutic method according to any one of (81) to (92) further comprising administering an antibody-drug conjugate, wherein the antibody specific for a tumor antigen is a part of the antibody-drug conjugate, and wherein the antibody-drug conjugate further comprises a linker and a drug, the antibody specific for a tumor antigen being connected to the drug via the linker, the linker and the drug forming a drug-linker.
(93a) A therapeutic method according to any one of (81) to (92) further comprising administering an antibody-drug conjugate wherein the antibody-drug conjugate comprises the antibody specific for a tumor antigen connected via a linker to a drug, the linker and the drug forming a drug-linker.
(94) A therapeutic method according to (93) or (93a), wherein the drug-linker is as represented by the following formula:

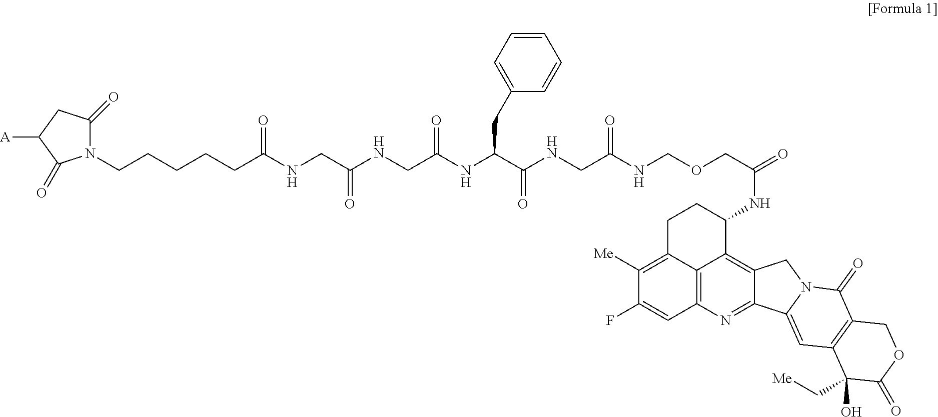

wherein A represents the connecting position to the antibody, and wherein the drug-linker is conjugated to the antibody specific for a tumor antigen via a thioether bond.
(95) A therapeutic method according to any one of (81) to (94), wherein the antibody specific for a tumor antigen is an anti-HER2 antibody, an anti-HER3 antibody, an anti-TROP2 antibody, an anti-B7-H3 antibody, an anti-GPR20 antibody, or an anti-CDH6 antibody.
(96) A therapeutic method according to (95), wherein the antibody specific for a tumor antigen is the anti-HER2 antibody.
(97) A therapeutic method according to (96), wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.
(98) A therapeutic method according to (96), wherein the anti-HER2 antibody is an antibody comprising a heavy chain consisting of an amino acid sequence represented by SEQ ID NO: 1 and a light chain consisting of an amino acid sequence represented by SEQ ID NO: 2.
(99) A therapeutic method according to any one of (96) to (98) as dependent on (94), wherein the average number of units of the drug-linker conjugated per antibody molecule in the antibody-drug conjugate is in the range of from 7 to 8.
(100) A therapeutic method according to (95), wherein the antibody specific for a tumor antigen is an anti-HER3 antibody.
(101) A therapeutic method according to (100), wherein the anti-HER3 antibody is an antibody comprising a heavy chain consisting of an amino acid sequence represented by SEQ ID NO: 3 and a light chain consisting of an amino acid sequence represented by SEQ ID NO: 4.
(102) A therapeutic method according to (101), wherein the anti-HER3 antibody lacks a lysine residue at the carboxyl terminus of the heavy chain.
(103) A therapeutic method according to any one of (100) to (102) as dependent on (94), wherein the average number of units of the drug-linker conjugated per antibody molecule in the antibody-drug conjugate is in the range of from 7 to 8.
(104) A therapeutic method according to (95), wherein the antibody specific for a tumor antigen is an anti-TROP2 antibody.
(105) A therapeutic method according to (104), wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.
(106) A therapeutic method according to (105), wherein the anti-TROP2 antibody lacks a lysine residue at the carboxyl terminus of the heavy chain.
(107) A therapeutic method according to any one of (104) to (106) as dependent on (94), wherein the average number of units of the drug-linker conjugated per antibody molecule in the antibody-drug conjugate is in the range of from 3.5 to 4.5.
(108) A therapeutic method according to (95), wherein the antibody specific for a tumor antigen is an anti-B7-H3 antibody.
(109) A therapeutic method according to (108), wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.
(110) A therapeutic method according to (109), wherein the anti-B7-H3 antibody lacks a lysine residue at the carboxyl terminus of the heavy chain.
(111) A therapeutic method according to any one of (108) to (110) as dependent upon (94), wherein the average number of units of the drug-linker conjugated per antibody molecule in the antibody-drug conjugate is in the range of from 3.5 to 4.5.
(112) A therapeutic method according to (95), wherein the antibody specific for a tumor antigen is an anti-GPR20 antibody.
(113) A therapeutic method according to (112), wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.
(114) A therapeutic method according to (113), wherein the anti-GPR20 antibody lacks a lysine residue at the carboxyl terminus of the heavy chain.
(115) A therapeutic method according to any one of (112) to (114) as dependent on (94), wherein the average number of units of the drug-linker conjugated per antibody molecule in the antibody-drug conjugate is in the range of from 7 to 8.
(116) A therapeutic method according to (95), wherein the antibody specific for a tumor antigen is an anti-CDH6 antibody.
(117) A therapeutic method according to (116), wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.
(118) A therapeutic method according to (117), wherein the anti-CDH6 antibody lacks a lysine residue at the carboxyl terminus of the heavy chain.
(119) A therapeutic method according to any one of (116) to (118) as dependent on (94), wherein the average number of units of the drug-linker conjugated per antibody molecule in the antibody-drug conjugate is in the range of from 7 to 8.
(120) A therapeutic method according to any one of (81) to (119), wherein the CD47 inhibitor and the antibody specific for a tumor antigen are separately contained active components in different formulations.
(121) A therapeutic method according to any one of (81) to (120), wherein the method is for treating at least one selected from the group consisting of: breast cancer, gastric cancer, colorectal cancer, lung cancer, esophageal cancer, salivary gland cancer, esophagogastric junction adenocarcinoma, biliary tract cancer, Paget's disease, pancreatic cancer, ovarian cancer, bladder cancer, prostate cancer, uterine carcinosarcoma, head and neck cancer, hepatocellular cancer, cervical cancer, brain tumor, glioma, eye tumor, thyroid cancer, thymus cancer, gallbladder cancer, lymphoma, leukemia, and myelodysplastic syndrome.
(122) An antibody specific for a tumor antigen for use in the treatment or prevention of cancer by simultaneous or sequential administration with a CD47 inhibitor.
(123) An antibody specific for a tumor antigen for use according to (122), wherein the CD47 inhibitor comprises SIRPα or a SIRPα derivative.
(124) An antibody specific for a tumor antigen for use according to (123), wherein the SIRPα derivative comprises a polypeptide having a sequence with at least 80%, 90%, 95%, 99% or 100% sequence identity to the residues 1 to 149 of SEQ ID NO: 15 or 16.
(125) An antibody specific for a tumor antigen for use according to claim 122) or (123), wherein the CD47 inhibitor is a fusion protein and further comprises an Fc region.
(126) An antibody specific for a tumor antigen for use according to (125), wherein the CD47 inhibitor comprises a polypeptide having a sequence with at least 80%, 90%, 95%, 99% or 100% sequence identity to SEQ ID NO: 15 or 16.
(127) An antibody specific for a tumor antigen for use according to any one of (122) to (126), wherein the CD47 inhibitor is Evorpacept.
(128) An antibody specific for a tumor antigen for use according to (123), wherein the CD47 inhibitor is selected from the group consisting of: TTI-621, TTI-622, DSP-107 and SL-172154.
(129) An antibody specific for a tumor antigen for use according to (122) wherein the CD47 inhibitor comprises an antibody or an antigen-binding fragment thereof, wherein the antibody is specific for CD47.
(130) An antibody specific for a tumor antigen for use according to (129), wherein the antibody specific for CD47 is selected from the group consisting of: Magrolimab, Lemsoparlimab, AO-176, SRF-231, IBI-188, IBI-322, IMC-002, MIL-95, TG-1801, ZL-1201, AK-117 (Ligufalimab) and IMM-0306.
(131) An antibody specific for a tumor antigen for use according to (122), wherein the CD47 inhibitor comprises a small-molecule agent capable of binding to CD47.
(132) An antibody specific for a tumor antigen for use according to (131), wherein the small molecule agent is selected from the group consisting of RRx-001 and IMM-01.
(133) An antibody specific for a tumor antigen for use according to any one of (122) to (132), wherein CD47 comprises a polypeptide sequence represented by SEQ ID NO: 13.
(134) An antibody specific for a tumor antigen for use according to any one of (122) to (133) wherein the antibody specific for a tumour antigen is part of an antibody-drug conjugate and wherein the antibody-drug conjugate comprises the antibody specific for a tumor antigen connected via a linker to a drug, the linker and the drug forming a drug-linker.
(135) An antibody specific for a tumor antigen for use according to (134), wherein drug-linker is as represented by the following formula:

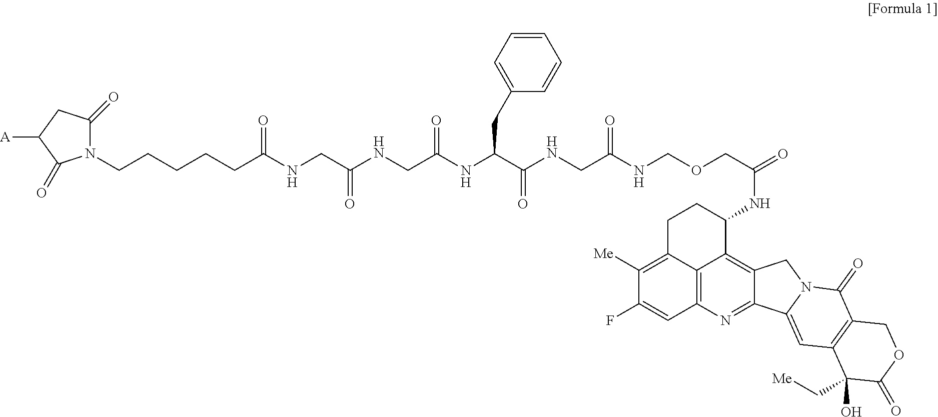

wherein A represents the connecting position to the antibody, and wherein the drug-linker is conjugated to the antibody specific for a tumor antigen via a thioether bond.
(136) An antibody specific for a tumor antigen for use according to any one of (133) to (135), wherein the antibody specific for a tumor antigen is an anti-HER2 antibody, an anti-HER3 antibody, an anti-TROP2 antibody, an anti-B7-H3 antibody, an anti-GPR20 antibody, or an anti-CDH6 antibody.
(137) An antibody specific for a tumor antigen for use according to (136), wherein the antibody specific for a tumor antigen is the anti-HER2 antibody.
(138) An antibody specific for a tumor antigen for use according to (137), wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.
(139) An antibody specific for a tumor antigen for use according to (137), wherein the anti-HER2 antibody is an antibody comprising a heavy chain consisting of an amino acid sequence represented by SEQ ID NO: 1 and a light chain consisting of an amino acid sequence represented by SEQ ID NO: 2.
(140) An antibody specific for a tumor antigen for use according to any one of (137) to (139) as dependent on (135), wherein the average number of units of the drug-linker conjugated per antibody molecule in the antibody-drug conjugate is in the range of from 7 to 8.
(141) An antibody specific for a tumor antigen for use according to (136), wherein the antibody specific for a tumor antigen is an anti-HER3 antibody.
(142) An antibody specific for a tumor antigen for use according to (141), wherein the anti-HER3 antibody is an antibody comprising a heavy chain consisting of an amino acid sequence represented by SEQ ID NO: 3 and a light chain consisting of an amino acid sequence represented by SEQ ID NO: 4.
(143) An antibody specific for a tumor antigen for use according to (142), wherein the anti-HER3 antibody lacks a lysine residue at the carboxyl terminus of the heavy chain.
(144) An antibody specific for a tumor antigen for use according to any one of (141) to (143) as dependent on (135), wherein the average number of units of the drug-linker conjugated per antibody molecule in the antibody-drug conjugate is in the range of from 7 to 8.
(145) An antibody specific for a tumor antigen for use according to (136), wherein the antibody specific for a tumor antigen is an anti-TROP2 antibody.
(146) An antibody specific for a tumor antigen for use according to (145), wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.
(147) An antibody specific for a tumor antigen for use according to (146), wherein the anti-TROP2 antibody lacks a lysine residue at the carboxyl terminus of the heavy chain.
(148) An antibody specific for a tumor antigen for use according to any one of (145) to (147) as dependent on (135), wherein the average number of units of the drug-linker conjugated per antibody molecule in the antibody-drug conjugate is in the range of from 3.5 to 4.5.
(149) An antibody specific for a tumor antigen for use according to (136), wherein the antibody specific for a tumor antigen is an anti-B7-H3 antibody.
(150) An antibody specific for a tumor antigen for use according to (149), wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.
(151) An antibody specific for a tumor antigen for use according to (150), wherein the anti-B7-H3 antibody lacks a lysine residue at the carboxyl terminus of the heavy chain.
(152) An antibody specific for a tumor antigen for use according to any one of (149) to (151) as dependent upon (135), wherein the average number of units of the drug-linker conjugated per antibody molecule in the antibody-drug conjugate is in the range of from 3.5 to 4.5.
(153) An antibody specific for a tumor antigen for use according to (136), wherein the antibody specific for a tumor antigen is an anti-GPR20 antibody.
(154) An antibody specific for a tumor antigen for use according to (153), wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.
(155) An antibody specific for a tumor antigen for use according to (154), wherein the anti-GPR20 antibody lacks a lysine residue at the carboxyl terminus of the heavy chain.
(156) An antibody specific for a tumor antigen for use according to any one of (153) to (155) as dependent on (135), wherein the average number of units of the drug-linker conjugated per antibody molecule in the antibody-drug conjugate is in the range of from 7 to 8.
(157) An antibody specific for a tumor antigen for use according to (136), wherein the antibody specific for a tumor antigen is an anti-CDH6 antibody.
(158) An antibody specific for a tumor antigen for use according to (157), wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.
(159) An antibody specific for a tumor antigen for use according to (158), wherein the anti-CDH6 antibody lacks a lysine residue at the carboxyl terminus of the heavy chain.
(160) An antibody specific for a tumor antigen for use according to any one of (157) to (159) as dependent on (135), wherein the average number of units of the drug-linker conjugated per antibody molecule in the antibody-drug conjugate is in the range of from 7 to 8.
(161) An antibody specific for a tumor antigen for use according to any one of (122) to (160) wherein the CD47 inhibitor and the antibody specific for a tumor antigen are separately contained active components in different formulations.
(162) An antibody specific for a tumor antigen for use according to any one of (122) to (161) wherein the cancer is at least one selected from the group consisting of: breast cancer, gastric cancer, colorectal cancer, lung cancer, esophageal cancer, salivary gland cancer, esophagogastric junction adenocarcinoma, biliary tract cancer, Paget's disease, pancreatic cancer, ovarian cancer, bladder cancer, prostate cancer, uterine cancer, sarcoma, head and neck cancer, hepatocellular cancer, cervical cancer, brain tumor, glioma, eye tumor, thyroid cancer, thymus cancer, gallbladder cancer, lymphoma, leukemia, and myelodysplastic syndrome.

### Advantageous Effects of Invention

The present invention can provide a pharmaceutical composition wherein a CD47 inhibitor and an antibody specific for a tumor antigen are administered in combination and/or a therapeutic method comprising administering a CD47 inhibitor and an antibody specific for a tumor antigen in combination to a subject.

### Definitions

In this specification, a “tumor antigen” is an antigenic substance which is produced in tumor cells. It includes both “tumor-specific antigens” (i.e. antigens which are present only on tumor cells and not on any other cell types) and “tumor-associated antigens” (i.e. antigens which are present on some tumor cells and also some normal cells but which are expressed at elevated levels on tumor cells). It includes HER2, HER3, an TROP2, B7-H3, GPR20 and CDH6.

In this specification, the term “SIRPα” means Signal regulatory protein α. In some embodiments, the amino acid sequence of SIRPα is the sequence of human SIRPα protein is disclosed in GenBank Accession No.: NP 001035111 and set forth in SEQ ID NO: 14.

In this specification, the term “CD47” means the Cluster of Differentiation 47 protein. In some embodiments, the amino acid sequence of CD47 is the sequence set forth in SEQ ID NO: 13.

In this specification, the term “small molecule” means an organic molecule having a low molecular weight such as less than 1000 Da.

In this specification, the term “antigen-binding fragment” of an antibody means a partial fragment of an antibody having an antigen-binding activity and includes Fab, F(ab′)2, scFv and the like. The term also encompasses Fab′ which is a monovalent fragment in a variable region of an antibody obtained by treating F(ab′)2 under reducing conditions. However, the term is not limited to these molecules as long as the fragment has a binding affinity for an antigen. Further, these antigen-binding fragments include not only a fragment obtained by treating a full-length molecule of an antibody protein with an appropriate enzyme, but also a protein produced in an appropriate host cell using a genetically modified antibody gene.

In this specification, the “identity” between two amino acid sequences has the following meaning. The identity between two amino acid sequences having completely identical amino acid sequences is 100%. Provided that one of the amino acid sequences has the substitution, deletion or addition of one or two or more amino acids or amino acid residues as compared with the other amino acid sequence, the identity between these two amino acid sequences is lower than 100%. Examples of an algorithm or a program for determining the identity between two sequences in consideration of a gap can include those known to a person skilled in the art, such as BLAST (Altschul, et al., Nucleic Acids Res., Vol. 25, p. 3389-3402, 1997), BLAST2 (Altschul, et al., J. Mol. Biol., Vol. 215, p. 403-410, 1990), and Smith-Waterman (Smith, et al., J. Mol. Biol., Vol. 147, p. 195-197, 1981).

In this specification, the term “antibody specific for” a target antigen means that the antibody binds to the target antigen preferentially as compared with unrelated target antigens.

### BRIEF DESCRIPTION OF DRAWINGS

FIG. 1 is a diagram showing an amino acid sequence of a heavy chain of an anti-HER2 antibody (SEQ ID NO: 1).

FIG. 2 is a diagram showing an amino acid sequence of a light chain of an anti-HER2 antibody (SEQ ID NO: 2).

FIG. 3 is a diagram showing an amino acid sequence of a heavy chain of an anti-HER3 antibody (SEQ ID NO: 3).

FIG. 4 is a diagram showing an amino acid sequence of a light chain of an anti-HER3 antibody (SEQ ID NO: 4).

FIG. 5 is a diagram showing an amino acid sequence of a heavy chain of an anti-TROP2 antibody (SEQ ID NO: 5).

FIG. 6 is a diagram showing an amino acid sequence of a light chain of an anti-TROP2 antibody (SEQ ID NO: 6).

FIG. 7 is a diagram showing an amino acid sequence of a heavy chain of an anti-B7-H3 antibody (SEQ ID NO: 7).

FIG. 8 is a diagram showing an amino acid sequence of a light chain of an anti-B7-H3 antibody (SEQ ID NO: 8).

FIG. 9 is a diagram showing an amino acid sequence of a heavy chain of an anti-GPR20 antibody (SEQ ID NO: 9).

FIG. 10 is a diagram showing an amino acid sequence of a light chain of an anti-GPR20 antibody (SEQ ID NO: 10).

FIG. 11 is a diagram showing an amino acid sequence of a heavy chain of an anti-CDH6 antibody (SEQ ID NO: 11).

FIG. 12 is a diagram showing an amino acid sequence of a light chain of an anti-CDH6 antibody (SEQ ID NO: 12).

FIG. 13 is a diagram showing an amino acid sequence of the CD47 protein (SEQ ID NO: 13).

FIG. 14 is a diagram showing an amino acid sequence of the SIRPα protein of GenBank Accession No.: NP 001035111 (SEQ ID NO: 14).

FIG. 15 is a diagram showing an amino acid sequence of the ALX148 hIgG1 construct.

FIG. 16 is a diagram showing an amino acid sequence of the ALX148 mIgG1 construct.

FIG. 17 is a graph showing the ADCP (%) activity of: (A) antibody-drug conjugate (1) in JIMT-1 cells and (B) antibody-drug conjugate (2) in AGS cells.

FIG. 18 is a graph showing the ADCP (%) activity of the antibody-drug conjugate (3) in NCI-H322 cells.

FIG. 19A is a diagram showing the treatment schedule of antibody-drug conjugate (1) and/or ALX148 mIgG1.

FIG. 19B is a summary of the number of animals with complete remission on Day 27 in the indicated treatment groups.

FIG. 20A is a diagram showing the treatment schedule of antibody-drug conjugate (2) and/or ALX148 mIgG1.

FIG. 20B is a graph showing tumor volume following inoculation with vehicle, ALX148 monotherapy, antibody-drug conjugate (2) monotherapy or antibody-drug conjugate (2) and ALX148 combination therapy.

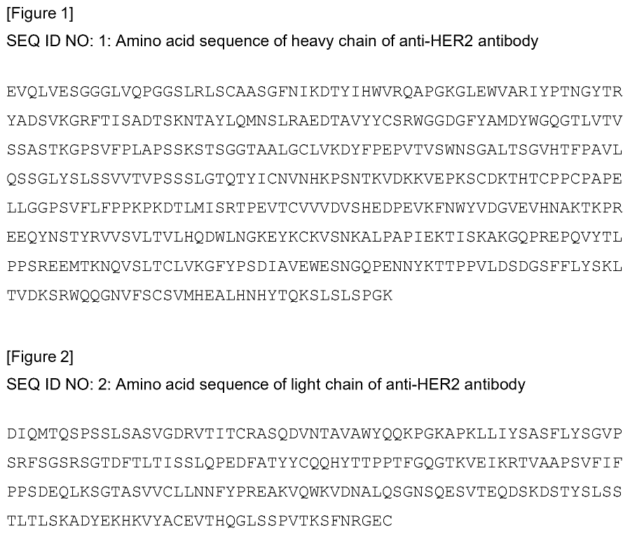

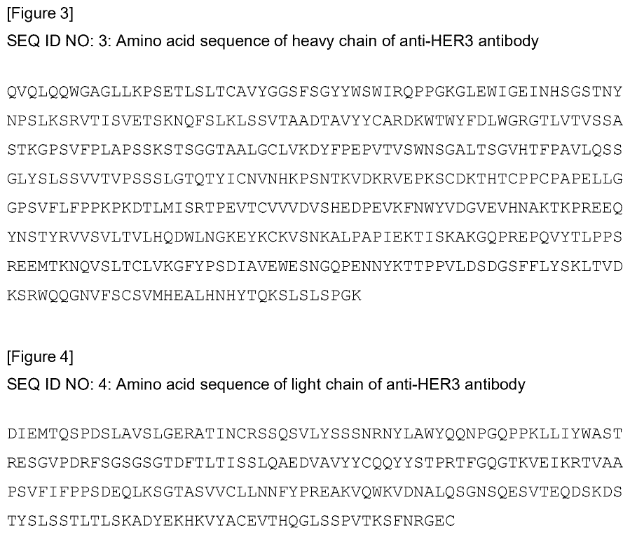

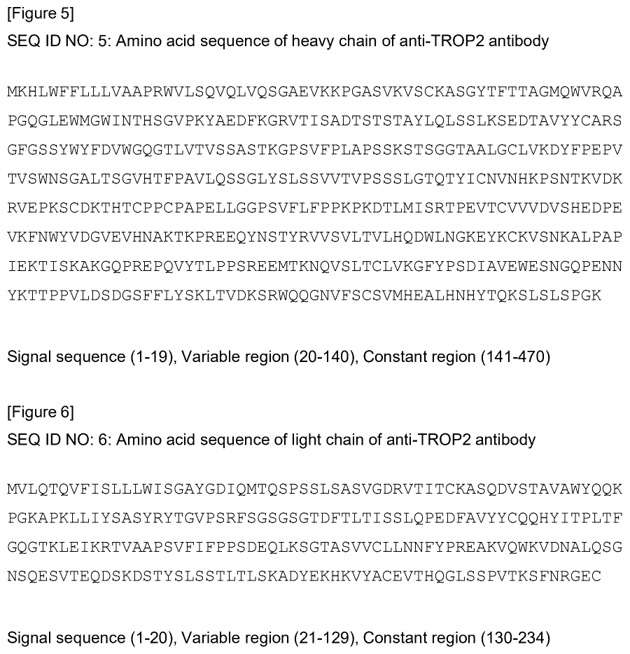

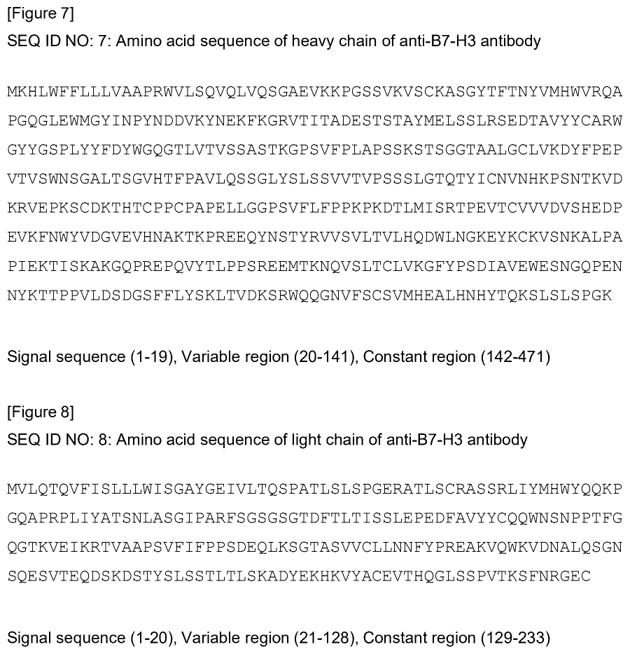

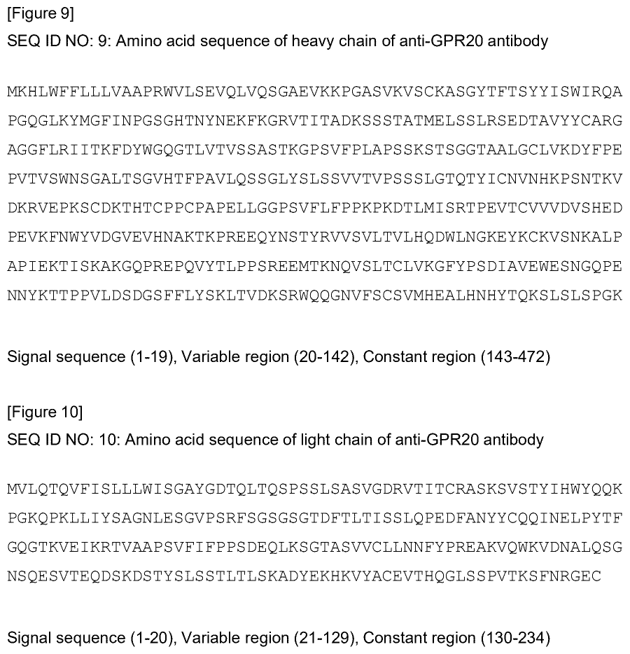

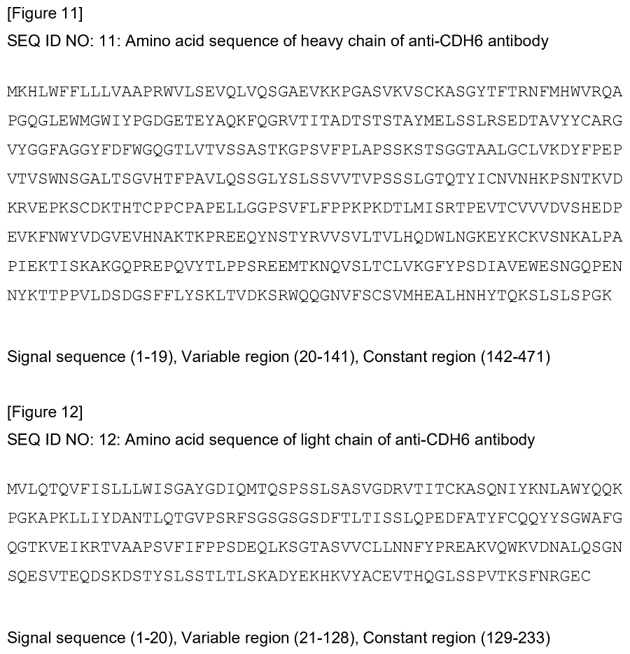

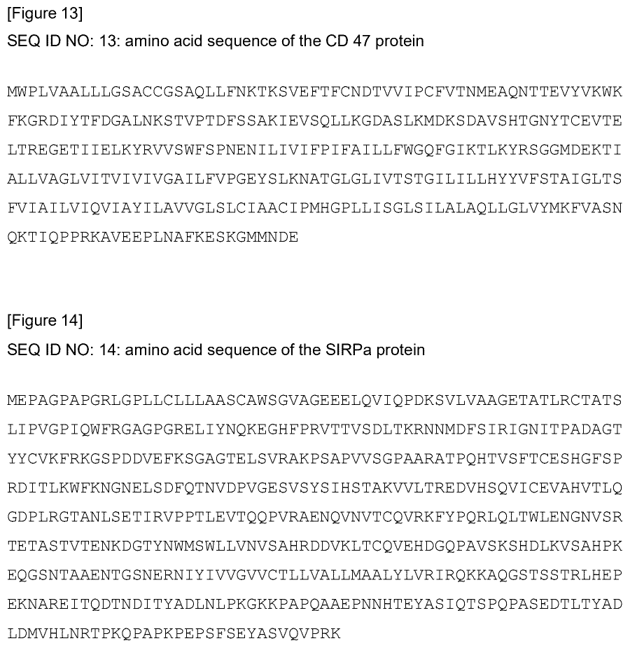

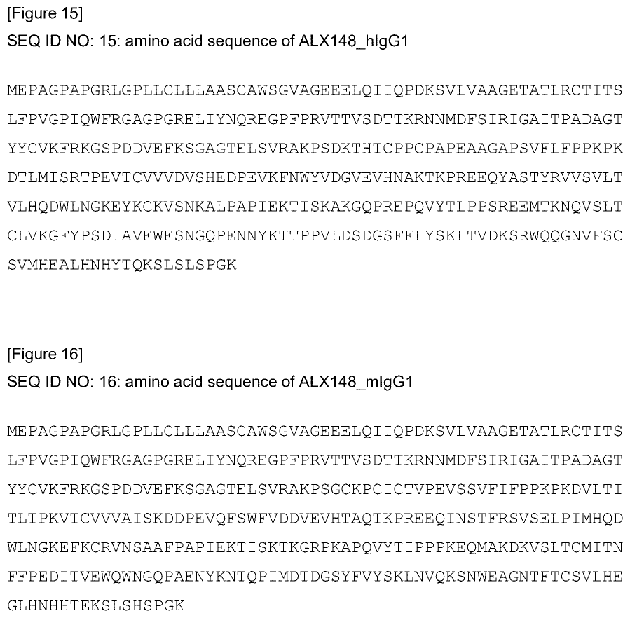

### DESCRIPTION OF EMBODIMENTS

Hereinafter, preferred modes for carrying out the present invention are described. The embodiments described below are given merely for illustrating one example of a typical embodiment of the present invention and are not intended to limit the scope of the present invention.

### 1. CD47 Inhibitor

The CD47 inhibitor is an agent that blocks binding between CD47 and SIRPα. Tumor cells highly express CD47. When SIRPα expressed on phagocytes having phagocytic activity binds to CD47 and they interact, a “Don't-eat-me” signal is transmitted to the phagocytes. In this way, the tumor cells escape from phagocytosis by the phagocytes. The CD47 inhibitor inhibits the binding between CD47 and SIRPα, thereby inhibiting transmission of a “Don't-eat-me” signal from tumor cells to phagocytes, thereby enhancing phagocytosis by phagocytes to engulf tumor cells. As a result, an antitumor effect can be exerted. Examples of phagocytes having phagocytic activity include macrophages such as M1 and M2 macrophages and dendritic cells such as immature dendritic cells (imDC).

In some embodiments of the present invention, the CD47 inhibitor is a polypeptide comprising the SIRPα protein. For example, in some embodiments, the CD47 inhibitor is a polypeptide comprising the sequence of SEQ ID NO: 14. In other embodiments, the CD47 inhibitor comprises a derivative of the SIRPα protein, that is to say, a protein that comprises a fragment of the SIRPα protein and/or which has at least 80%, 90%, 95% or 99% sequence identity to SEQ ID NO: 14. The derivatives of the SIRPα protein retain the binding specificity to the CD47 protein. For example, in some embodiments, the derivative of the SIRPα protein comprises the D1 domain of the SIRPα protein in particular, in one example, the CD47 inhibitor comprises a polypeptide having at least 80%, 90%, 95%, 99% or 100% sequence identity to the residues 1 to 149 of SEQ ID NO: 15 or 16.

In some alternative embodiments, the CD47 inhibitor is a fusion protein and comprises the SIRPα protein or derivative thereof, linked to a further polypeptide at the N-terminus or the C-terminus of the SIRPα protein or derivative thereof. In preferred embodiments, the further polypeptide comprises a region of an Fc domain. In some variants of this embodiment, the Fc domain is mutated as compared with the wild-type version thereof in order to ablate or reduce binding to an Fcγ receptor. In some embodiments comprising the SIRPA protein and the Fc domain, the SIRPα protein is directly conjugated to the Fc domain whereas in other embodiments, the SIRPα protein is connected to the Fc domain via a linker (e.g. a spacer) therebetween. In some further embodiments, the CD47 inhibitor is further linked to one or more additional components such as a polymer (e.g. a PEG polymer) in order to improve the pharmacokinetic properties thereof.

In a particularly preferred embodiment, the CD47 inhibitor comprises a polypeptide comprising a sequence of SEQ ID NO: 15. It is to be appreciated that amino acid residues 1 to 149 of SEQ ID NO: 15 correspond to the SIRPα protein and the region of residues 150 to 376 of SEQ ID NO: 15 correspond to the human IgG1 Fc protein. Therefore, in some variants of this embodiment, the CD47 inhibitor comprises a polypeptide having a first region having at least 80%, 90%, 95% or 99% sequence identity to the residues 1 to 149 of SEQ ID NO: 15 linked to a second region having at least 80%, 90%, 95% or 99% sequence identity to the residues 150 to 376 of SEQ ID NO: 15 and which retains binding to the CD47 protein.

In another preferred embodiment, the CD47 inhibitor comprises a polypeptide comprising a sequence of SEQ ID NO: 16. It is to be appreciated that amino acid residues 1 to 149 of SEQ ID NO: 16 correspond to the SIRPα protein and the region of residues 150 to 371 of SEQ ID NO: 16 correspond to the mouse IgG1 Fc protein. Therefore, in some variants of this embodiment, the CD47 inhibitor comprises a polypeptide having a first region having at least 80%, 90%, 95% or 99% sequence identity to the residues 1 to 149 of SEQ ID NO: 16 linked to a second region having at least 80%, 90%, 95% or 99% sequence identity to the residues 150 to 371 of SEQ ID NO: 16 and which retains binding to the CD47 protein.

In a particularly preferred embodiment, the CD47 inhibitor is Evorpacept, which is also known as ALX148.

In still further embodiments, the CD47 inhibitor is one of TTI-621 (WO2014/094122), TTI-622 (WO2014/094122), DSP-107 (WO2018/127919) and SL-172154.

In other embodiments of the present invention, the CD47 inhibitor is a small molecule agent. In particular embodiments, the small molecule agent is RRx-001 (J. Med. Chem. 2021, 64, 11, 7261-7271) or IMM-01.

In some embodiments, the CD47 inhibitor is an anti-CD47 antibody. In such embodiments, the antibody can be obtained in the same manner as described in “2. Antibody Specific for the Tumor Antigen”.

In particular, the anti-CD47 antibody which is a monoclonal antibody can be obtained by immunizing a mammal such as a mouse, a rat, a rabbit, a hamster, a guinea pig, a horse, a monkey, a dog, a pig, a cow, a goat, a sheep, with CD47 or a fragment thereof used as an immunogen, fusing the spleen cells and myeloma cells to obtain hybridoma and allowing the hybridoma to produce and secrete the antibody. The hybridoma can be produced by a method known in the art.

CD47 serving as an immunogen can be chemically synthesized based on sequence information or can be obtained as a recombinant protein, which is produced based on a DNA sequence encoding a protein and in accordance with a method known in the art.

The antibody can be screened in any method; preferably, by Cell-ELISA transfected with DNA encoding CD47.

In the anti-CD47 antibody used in embodiments of the present invention, modified variants of the antibody are also included. The modified variant refers to a variant obtained by subjecting the anti-CD47 antibody to be used in the present invention to chemical or biological modification. Examples of the chemically modified variant include variants having a linkage of a chemical moiety to an amino acid skeleton, and variants having a linkage of a chemical moiety to an N-linked or O-linked carbohydrate chain. Examples of the biologically modified variant include variants obtained by post-translational modification (such as N-linked or O-linked glycosylation, N- or C-terminal processing, deamidation, isomerization of aspartic acid, or oxidation of methionine), and variants in which a methionine residue has been added to the N terminus by expression using a prokaryotic host cell. Further, an antibody labeled so as to enable the detection or isolation of the anti-CD47 antibody used in such embodiments of the present invention, for example, an enzyme-labeled antibody, a fluorescence-labeled antibody and an affinity-labeled antibody are also included in the meaning of the modified variant. Such a modified variant of the anti-CD47 antibody used in the present invention is useful for, e.g., improving the stability and blood retention of the antibody, reducing the antigenicity thereof, or detecting or isolating the antibody.

Note that, it is known that a lysine residue at the carboxyl terminus of the heavy chain of an antibody produced in a cultured mammalian cell is deleted (Journal of Chromatography A, 705:129-134 (1995)). It is also known that two amino acid residues (glycine and lysine) at the carboxyl terminus of the heavy chain of an antibody produced in a cultured mammalian cell are deleted and a proline residue newly located at the carboxyl terminus is amidated (Analytical Biochemistry, 360:75-83 (2007)). However, such deletion and modification of the heavy chain sequence do not affect the antigen-binding affinity and the effector function (e.g., activation of complement, antibody-dependent cellular cytotoxicity,) of the antibody. Therefore, in the anti-CD47 antibody used in embodiments of the present invention, antibodies subjected to such modification and functional fragments thereof are also included, and deletion variants in which one or two amino acids have been deleted at the carboxyl terminus of the heavy chain, deletion variants having an amidated residue (for example, a heavy chain in which the carboxyl-terminal proline residue has been amidated) are also included. Note that the type of deletion variant having a deletion at the carboxyl terminus of the heavy chain of the anti-CD47 antibody used in embodiments of the present invention is not limited to the above variants as long as the antigen-binding affinity and the effector function are conserved. The two heavy chains constituting the anti-CD47 antibody used in the present invention may be one selected from the group consisting of a full-length heavy chain and the above-described heavy chain having a deletion, or may be of two types in combination selected therefrom. The ratio of the amount of each deletion variant can be affected by the type of cultured mammalian cells which produce the anti-CD47 antibody used in the present invention and the culture conditions; however, an antibody in which one amino acid residue at the carboxyl terminus has been deleted in both of the two heavy chains in the anti-CD47 antibody used in the present invention can be preferably exemplified.

The anti-CD47 antibody used in the present invention includes a chimeric antibody modified in order to decline heterogeneous antigenicity to humans and a humanized antibody. The humanized antibody is also referred to as a CDR-transplanted antibody.

The chimeric antibody refers to an antibody consisting of a light chain variable region and a heavy chain variable region of an antibody of a non-human animal, and a light chain constant region and a heavy chain constant region of a human antibody. The chimeric antibody can be prepared by taking cDNA encoding a light chain variable region and cDNA encoding a heavy chain variable region from a hybridoma producing an anti-CD47 antibody, and inserting the cDNAs into an expression vector having cDNA encoding a light chain constant region and a heavy chain constant region of a human antibody to construct a chimeric antibody expression vector, introducing the chimeric antibody expression vector into host cells and allowing expression of the antibody.

It is known that in an antibody produced in a cultured mammalian cell, a lysine residue at the carboxyl terminus of the heavy chain is deleted (Tsubaki et. al., Int. J. Biol. Macromol, 139-147, 2013). However, the deletion of the heavy chain sequence does not affect the antigen-binding affinity and the effector function (e.g. activation of a complement and antibody-dependent cellular cytotoxicity) of the antibody. Thus, in the present invention, an antibody lacking a lysine residue at the carboxyl terminus of the heavy chain is included.

Specific examples of an anti-CD47 antibody include: Magrolimab (INN RN: 2169232-81-7), Lemsoparlimab (INN RN: 2377483-71-9), AO-176 (WO20198370), SRF-231 (WO18236904), IBI-188, IBI-322, IMC-002, MIL-95, TG-1801, ZL-1201, AK-117 (Ligufalimab) and IMM-0306.

### 2. Antibody Specific for the Tumor Antigen

The antibody specific for the tumor antigen, used in the present invention may be derived from any species and is preferably an antibody derived from a human, a rat, a mouse, or a rabbit. In cases when the antibody is derived from species other than human species, it is preferably chimerized or humanized using a well-known technique. The antibody specific for the tumor antigen may be a polyclonal antibody or a monoclonal antibody and is preferably a monoclonal antibody.

The antibody specific for the tumor antigen is an antibody having a characteristic of being capable of targeting cancer cells and is preferably an antibody possessing, for example, a property of recognizing a cancer cell, a property of binding to a cancer cell, a property of internalizing in a cancer cell, and/or cytocidal activity against cancer cells.

The binding activity of the antibody against cancer cells can be using confirmed flow cytometry. The internalization of the antibody into tumor cells can be confirmed using (1) an assay of visualizing an antibody incorporated in cells under a fluorescence microscope using a secondary antibody (fluorescently labeled) binding to the therapeutic antibody (Cell Death and Differentiation (2008) 15, 751-761), (2) an assay of measuring a fluorescence intensity incorporated in cells using a secondary antibody (fluorescently labeled) binding to the therapeutic antibody (Molecular Biology of the Cell, Vol. 15, 5268-5282 December 2004), or (3) a Mab-ZAP assay using an immunotoxin binding to the therapeutic antibody wherein the toxin is released upon incorporation into cells to inhibit cell growth (Bio Techniques 28:162-165, January 2000). As the immunotoxin, a recombinant complex protein of a diphtheria toxin catalytic domain and protein G may be used.

The antitumor activity of the antibody can be confirmed in vitro by determining inhibitory activity against cell growth. For example, a cancer cell line overexpressing a target protein for the antibody is cultured, and the antibody is added at varying concentrations into the culture system to determine inhibitory activity against focus formation, colony formation, and spheroid growth. The antitumor activity can be confirmed in vivo, for example, by administering the antibody to a nude mouse with a transplanted cancer cell line highly expressing the target protein, and determining change in the cancer cell.

It is preferred but not essential that the antibody specific for the tumor antigen should itself have an antitumor effect. In embodiments in which the antibody specific for the tumor antigen is part of an antibody-drug conjugate, there is the additional consideration that, for the purpose of specifically and selectively exerting the cytotoxic activity of the antitumor compound against cancer cells, it is important and also preferred that the antibody should have the property of internalizing to migrate into cancer cells.

The antibody specific for the tumor antigen used in the present invention can be obtained by a procedure known in the art. For example, the antibody can be obtained using a method usually carried out in the art, which involves immunizing animals with an antigenic polypeptide (i.e. the tumor antigen) and collecting and purifying antibodies produced in vivo. The origin of the antigen is not limited to humans, and the animals may be immunized with an antigen derived from a non-human animal such as a mouse, a rat and the like. In this case, the cross-reactivity of antibodies binding to the obtained heterologous antigen with human antigens can be tested to screen for an antibody applicable to a human disease.

Alternatively, antibody-producing cells which produce antibodies against the antigen are fused with myeloma cells according to a method known in the art (for example, Kohler and Milstein, Nature (1975) 256, p. 495-497; Kennet, R. ed., Monoclonal Antibodies, p. 365-367, Plenum Press, N.Y. (1980)), to establish hybridomas, from which monoclonal antibodies can, in turn, be obtained.

The antigen can be obtained by genetically engineering host cells to produce a gene encoding the antigenic protein. Specifically, vectors that permit expression of the antigen gene are prepared and transferred to host cells so that the gene is expressed. The antigen thus expressed can be purified. The antibody can also be obtained by a method of immunizing animals with the above-described genetically engineered antigen-expressing cells or a cell line expressing the antigen.

The antibody specific for the tumor antigen is preferably a recombinant antibody obtained by artificial modification for the purpose of decreasing heterologous antigenicity to humans such as a chimeric antibody or a humanized antibody, or is preferably an antibody having only the gene sequence of an antibody derived from a human, that is, a human antibody. These antibodies can be produced using a known method.

As the chimeric antibody, an antibody in which antibody variable and constant regions are derived from different species, for example, a chimeric antibody in which a mouse- or rat-derived antibody variable region is connected to a human-derived antibody constant region can be exemplified (Proc. Natl. Acad. Sci. USA, 81, 6851-6855, (1984)).

As the humanized antibody, an antibody obtained by integrating only the complementarity determining region (CDR) of f a heterologous antibody into a human-derived antibody (Nature (1986) 321, pp. 522-525), and an antibody obtained by grafting a part of the amino acid residues of the framework of a heterologous antibody as well as the CDR sequence of the heterologous antibody to a human antibody by a CDR-grafting method (WO 90/07861), and an antibody humanized using a gene conversion mutagenesis strategy (U.S. Pat. No. 5,821,337) can be exemplified.

As the human antibody, an antibody generated by using a human antibody-producing mouse having a human chromosome fragment including genes of a heavy chain and light chain of a human antibody (see Tomizuka, K. et al., Nature Genetics (1997) 16, p. 133-143; Kuroiwa, Y. et. al., Nucl. Acids Res. (1998) 26, p. 3447-3448; Yoshida, H. et. al., Animal Cell Technology: Basic and Applied Aspects vol. 10, p. 69-73 (Kitagawa, Y., Matsuda, T. and Iijima, S. eds.), Kluwer Academic Publishers, 1999; Tomizuka, K. et. al., Proc. Natl. Acad. Sci. USA (2000) 97, p. 722-727, etc.) can be exemplified. As an alternative, an antibody obtained by phage display, the antibody being selected from a human antibody library (see Wormstone, I. M. et. al, Investigative Ophthalmology & Visual Science. (2002) 43 (7), p. 2301-2308; Carmen, S. et. al., Briefings in Functional Genomics and Proteomics (2002), 1 (2), p. 189-203; Siriwardena, D. et. al., Ophthalmology (2002) 109 (3), p. 427-431, etc.) can be exemplified.

In the antibody specific for the tumor antigen, modified variants of the antibody are also included. The modified variant refers to a variant obtained by subjecting the antibody specific for the tumor antigen to chemical or biological modification. Examples of the chemically modified variant include variants including a linkage of a chemical moiety to an amino acid skeleton, variants including a linkage of a chemical moiety to an N-linked or O-linked carbohydrate chain, etc. Examples of the biologically modified variant include variants obtained by post-translational modification (such as N-linked or O-linked glycosylation, N- or C-terminal processing, deamidation, isomerization of aspartic acid, or oxidation of methionine), and variants in which a methionine residue has been added to the N terminus by being expressed in a prokaryotic host cell. Further, an antibody labeled so as to enable the detection or isolation of the antibody or an antigen according to the present invention, for example, an enzyme-labeled antibody, a fluorescence-labeled antibody, and an affinity-labeled antibody are also included in the meaning of the modified variant. Such a modified variant of the antibody specific for the tumor antigen is useful for improving the stability and blood retention of the antibody, reducing the antigenicity thereof, detecting or isolating an antibody or an antigen, and so on.

Further, by regulating the modification of a glycan which is linked to the antibody specific for the tumor antigen (glycosylation, defucosylation, etc.), it is possible to enhance antibody-dependent cellular cytotoxic activity. As the technique for regulating the modification of a glycan of antibodies, International Publication No. WO 99/54342, International Publication No. WO 00/61739, International Publication No. WO 02/31140, International Publication No. WO 2007/133855, and International Publication No. WO 2013/120066, etc. are known. However, the technique is not limited thereto. In the antibody specific for the tumor antigen, antibodies in which the modification of a glycan is regulated are also included.

It is known that a lysine residue at the carboxyl terminus of the heavy chain of an antibody produced in a cultured mammalian cell deleted is (Journal of Chromatography A, 705:129-134 (1995)), and it is also known that two amino acid residues (glycine and lysine) at the carboxyl terminus of the heavy chain of an antibody produced in a cultured mammalian cell are deleted and a proline residue newly located at the carboxyl terminus is amidated (Analytical Biochemistry, 360:75-83 (2007)). However, such deletion and modification of the heavy chain sequence do not affect the antigen-binding affinity and the effector function (the activation of complement, antibody-dependent cellular cytotoxicity, etc.) of the antibody. Therefore, in the antibody specific for the tumor antigen, antibodies subjected to such modification and functional fragments of the antibody are also included, and deletion variants in which one or two amino acids have been deleted at the carboxyl terminus of the heavy chain, deletion variants having an amidated residue (for example, a heavy chain in which the carboxyl-terminal proline residue has been amidated), and the like are also included. The type of deletion variant having a deletion at the carboxyl terminus of the heavy chain of the antibody according to the present invention is not limited to the above variants as long as the antigen-binding affinity and the effector function are conserved. The two heavy chains constituting the antibody specific for the tumor antigen may be of one type selected from the group consisting of a full-length heavy chain and the above-described deletion variant, or may be of two types in combination selected therefrom. The ratio of the amount of each deletion variant can be affected by the type of cultured mammalian cells which produce the antibody according to the present invention and the culture conditions; however, an antibody in which one amino acid residue at the carboxyl terminus has been deleted in both of the two heavy chains in the antibody specific for a tumor antigen can be preferably exemplified.

As isotypes of the antibody specific for the tumor antigen, for example, IgG (IgG1, IgG2, IgG3, IgG4) can be exemplified, and IgG1, IgG2 or IgG4 can be exemplified preferably.

Examples of antibodies specific for a tumor antigen can include, but are not particularly limited to, an anti-HER2 antibody, an anti-HER3 antibody, an anti-TROP2 antibody, an anti-B7-H3 antibody, an anti-CD3 antibody, an anti-CD30 antibody, an anti-CD33 antibody, an anti-CD37 antibody, an anti-CD56 antibody, an anti-CD98 antibody, an anti-DR5 antibody, an anti-EGFR antibody, an anti-EPHA2 antibody, an anti-FGFR2 antibody, an anti-FGFR4 antibody, an anti-FOLR1 antibody, an anti-VEGF antibody, an anti-CD20 antibody, an anti-CD22 antibody, an anti-CD70 antibody, an anti-PSMA antibody, an anti-CEA antibody, an anti-Mesothelin antibody, an anti-A33 antibody, an anti-CanAg antibody, an anti-Cripto antibody, an anti-G250 antibody, an anti-MUC1 antibody, an anti-GPNMB antibody, an anti-Integrin antibody, an anti-Tenascin-C antibody, an anti-SLC44A4 antibody, an anti-GPR20 antibody, and an anti-CDH6 antibody, and an anti-HER2 antibody, an anti-HER3 antibody, an anti-TROP2 antibody, an anti-B7-H3 antibody, an anti-GPR20 antibody, and an anti-CDH6 antibody can be preferably exemplified.

In some embodiments, the antibody specific for a tumor-associated antigen comprises multiple antibodies, each antibody specific for a different tumor antigen. For example, in some embodiments, two antibodies are provided, each specific for a different tumor-associated antigen. More generally, in some embodiments, there are provided multiple antibodies, each antibody specific for a different tumor antigen and each antibody independently selected from the following group: an anti-HER2 antibody, an anti-HER3 antibody, an anti-TROP2 antibody, an anti-B7-H3 antibody, an anti-CD3 antibody, an anti-CD30 antibody, an anti-CD33 antibody, an anti-CD37 antibody, an anti-CD56 antibody, an anti-CD98 antibody, an anti-DR5 antibody, an anti-EGFR antibody, an anti-EPHA2 antibody, an anti-FGFR2 antibody, an anti-FGFR4 antibody, an anti-FOLR1 antibody, an anti-VEGF antibody, an anti-CD20 antibody, an anti-CD22 antibody, an anti-CD70 antibody, an anti-PSMA antibody, an anti-CEA antibody, an anti-Mesothelin antibody, an anti-A33 antibody, an anti-CanAg antibody, an anti-Cripto antibody, an anti-G250 antibody, an anti-MUC1 antibody, an anti-GPNMB antibody, an anti-Integrin antibody, an anti-Tenascin-C antibody, an anti-SLC44A4 antibody, an anti-GPR20 antibody, and an anti-CDH6 antibody, and an anti-HER2 antibody, an anti-HER3 antibody, an anti-TROP2 antibody, an anti-B7-H3 antibody, an anti-GPR20 antibody, and an anti-CDH6 antibody.

In the present specification, the term “anti-HER2 antibody” refers to an antibody which specifically binds to HER2 (Human Epidermal Growth Factor Receptor Type 2; ErbB-2), and preferably has an activity of internalizing in HER2-expressing cells by binding to HER2.

Examples of the anti-HER2 antibody include trastuzumab (U.S. Pat. No. 5,821,337) and pertuzumab (International Publication No. WO 01/00245), and trastuzumab can be preferably exemplified.

An anti-HER2 antibody is preferably an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1 and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2 and CDRL3 consisting of an amino acid sequence consisting of amino acid residues 89 to 97 of SEQ ID NO: 2, and more preferably an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2, and even more preferably an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2; or an antibody comprising a heavy chain consisting of an amino acid sequence represented by SEQ ID NO: 1 and a light chain consisting of an amino acid sequence represented by SEQ ID NO: 2.

In the present specification, the term “anti-HER3 antibody” refers to an antibody which specifically binds to HER3 (Human Epidermal Growth Factor Receptor Type 3; ErbB-3), and preferably has an activity of internalizing in HER3-expressing cells by binding to HER3.

Examples of the anti-HER3 antibody include patritumab (U3-1287), (International U1-59 Publication No. WO 2007/077028), MM-121 (seribantumab), an anti-ERBB3 antibody described in International Publication No. WO 2008/100624, RG-7116 (lumretuzumab), and LJM-716 (elgemtumab), and patritumab and U1-59 can be preferably exemplified.

The anti-HER3 antibody is preferably an antibody comprising a heavy chain consisting of CDRH1 consisting of an amino acid sequence consisting of amino acid residues 26 to 35 of SEQ ID NO: 3, CDRH2 consisting of an amino acid sequence consisting of amino acid residues 50 to 65 of SEQ ID NO: 3, and CDRH3 consisting of an amino acid sequence consisting of amino acid residues 98 to 106 of SEQ ID NO: 3, and a light chain consisting of CDRL1 consisting of an amino acid sequence consisting of amino acid residues 24 to 39 of SEQ ID NO: 4, CDRL2 consisting of an amino acid sequence consisting of amino acid residues 56 to 62 of SEQ ID NO: 4, and CDRL3 consisting of an amino acid sequence consisting of amino acid residues 95 to 103 of SEQ ID NO: 4;
more preferably, an antibody comprising a heavy chain which comprises a heavy chain variable region consisting of an amino acid sequence consisting of amino acid residues 1 to 117 of SEQ ID NO: 3 and a light chain which comprises a light chain variable region consisting of an amino acid sequence consisting of amino acid residues 1 to 113 of SEQ ID NO: 4; and
even more preferably, an antibody comprising a heavy chain consisting of the amino acid sequence represented by SEQ ID NO: 3 and a light chain consisting of the amino acid sequence represented by SEQ ID NO: 4, or a variant of the antibody in which a lysine residue at the carboxyl terminus of the heavy chain is deleted.

In the present specification, the term “anti-TROP2 antibody” refers to an antibody which specifically binds to TROP2 (TACSTD2: Tumor calcium signal transducer 2; EGP-1), and preferably has an activity of internalizing in TROP2-expressing cells by binding to TROP2.

Examples of the anti-TROP2 antibody include hTINA1-H1L1 (International Publication No. WO 2015/098099).

The anti-TROP2 antibody is preferably an antibody comprising a heavy chain consisting of CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5, and a light chain consisting of CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6;
more preferably, an antibody comprising a heavy chain which comprises a heavy chain variable region consisting of an amino acid sequence consisting of amino acid residues 20 to 140 of SEQ ID NO: 5 and a light chain which comprises a light chain variable region consisting of an amino acid sequence consisting of amino acid residues 21 to 129 of SEQ ID NO: 6; and
even more preferably, an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6, or a variant of the antibody in which a lysine residue at the carboxyl terminus of the heavy chain is deleted.

In the present specification, the term 1 “anti-B7-H3 antibody” refers to an antibody which specifically binds to B7-H3 (B cell antigen #7 homolog 3; PD-L3; CD276), and preferably has an activity of internalizing in B7-H3-expressing cells by binding to B7-H3.

Examples of the anti-B7-H3 antibody include M30-H1-L4 (International Publication No. WO 2014/057687).

The anti-B7-H3 antibody is preferably an antibody comprising a heavy chain consisting of CDRH1 consisting of an amino acid sequence consisting of amino acid residues 50 to 54 of SEQ ID NO: 7, CDRH2 consisting of an amino acid sequence consisting of amino acid residues 69 to 85 of SEQ ID NO: 7, and CDRH3 consisting of an amino acid sequence consisting of amino acid residues 118 to 130 of SEQ ID NO: 7, and a light chain consisting of CDRL1 consisting of an amino acid sequence consisting of amino acid residues 44 to 53 of SEQ ID NO: 8, CDRL2 consisting of an amino acid sequence consisting of amino acid residues 69 to 75 of SEQ ID NO: 8, and CDRL3 consisting of an amino acid sequence consisting of amino acid residues 108 to 116 of SEQ ID NO: 8;
more preferably, an antibody comprising a heavy chain which comprises a heavy chain variable region consisting of an amino acid sequence consisting of amino acid residues 20 to 141 of SEQ ID NO: 7 and a light chain which comprises a light chain variable region consisting of an amino acid sequence consisting of amino acid residues 21 to 128 of SEQ ID NO: 8; and
even more preferably, an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8, or a variant of the antibody in which a lysine residue at the carboxyl terminus of the heavy chain is deleted.

In the present specification, the term “anti-GPR20 antibody” refers to an antibody which specifically binds to GPR20 (G Protein-coupled receptor 20), and preferably has an activity of internalizing in GPR20-expressing cells by binding to GPR20.

Examples of the anti-GPR20 antibody include h046-H4e/L7 (International Publication No. WO 2018/135501).

The anti-GPR20 antibody is preferably an antibody comprising a heavy chain consisting of CDRH1 consisting of an amino acid sequence consisting of amino acid residues 45 to 54 of SEQ ID NO: 9, CDRH2 consisting of an amino acid sequence consisting of amino acid residues 69 to 78 of SEQ ID NO: 9, and CDRH3 consisting of an amino acid sequence consisting of amino acid residues 118 to 131 of SEQ ID NO: 9, and a light chain consisting of CDRL1 consisting of an amino acid sequence consisting of amino acid residues 44 to 54 of SEQ ID NO: 10, CDRL2 consisting of an amino acid sequence consisting of amino acid residues 70 to 76 of SEQ ID NO: 10, and CDRL3 consisting of an amino acid sequence consisting of amino acid residues 109 to 117 of SEQ ID NO: 10;
more preferably, an antibody comprising a heavy chain which comprises a heavy chain variable region consisting of an amino acid sequence consisting of amino acid residues 20 to 142 of SEQ ID NO: 9 and a light chain which comprises a light chain variable region consisting of an amino acid sequence consisting of amino acid residues 21 to 129 of SEQ ID NO: 10; and
even more preferably, an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10, or a variant of the antibody in which a lysine residue at the carboxyl terminus of the heavy chain is deleted.

In the present specification, the term “anti-CDH6 antibody” refers to an antibody which specifically binds to CDH6 (Cadherin-6), and preferably has an activity of internalizing in CDH6-expressing cells by binding to CDH6.

Examples of the anti-CDH6 antibody include H01L02 (International Publication No. WO 2018/212136).

The anti-CDH6 antibody is preferably an antibody comprising a heavy chain consisting of CDRH1 consisting of an amino acid sequence consisting of amino acid residues 45 to 54 of SEQ ID NO: 11, CDRH2 consisting of an amino acid sequence consisting of amino acid residues 69 to 78 of SEQ ID NO: 11, and CDRH3 consisting of an amino acid sequence consisting of amino acid residues 118 to 130 of SEQ ID NO: 11, and a light chain consisting of CDRL1 consisting of an amino acid sequence consisting of amino acid residues 44 to 54 of SEQ ID NO: 12, CDRL2 consisting of an amino acid sequence consisting of amino acid residues 70 to 76 of SEQ ID NO: 12 and CDRL3 consisting of an amino acid sequence consisting of amino acid residues 109 to 116 of SEQ ID NO: 12;
more preferably, an antibody comprising a heavy chain which comprises a heavy chain variable region consisting of an amino acid sequence consisting of amino acid residues 20 to 141 of SEQ ID NO: 11 and a light chain which comprises a light chain variable region consisting of an amino acid sequence consisting of amino acid residues 21 to 128 of SEQ ID NO: 12; and
even more preferably, an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12, or a variant of the antibody in which a lysine residue at the carboxyl terminus of the heavy chain is deleted.

### 3. Antibody-Drug Conjugate

In some embodiments of the present invention, the antibody specific for the tumor antigen is part of an antibody-drug conjugate comprising the antibody specific for the tumor antigen connected via a linker to a drug. The partial structure consisting of the linker and the drug in the antibody-drug conjugate is referred to as a “drug-linker”.

It is particularly preferred that the antibody-drug conjugate is an antibody-drug conjugate in which a drug-linker represented by the following formula:

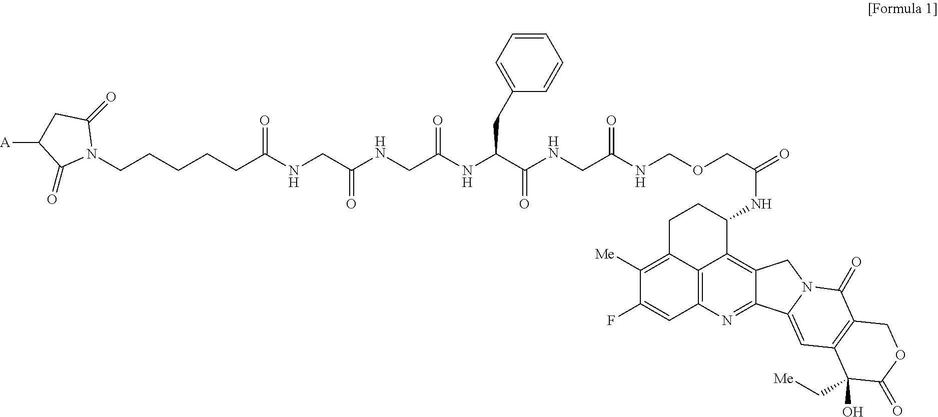

wherein A represents the connecting position to an antibody,
   is conjugated to the antibody specific for the tumor antigen via a thioether bond.

The drug-linker is connected to a thiol group (in other words, the sulfur atom of a cysteine residue) formed at an interchain disulfide bond site (two sites between heavy chains, and two sites between a heavy chain and a light chain) in the antibody.

The drug-linker of embodiments includes exatecan (IUPAC name: (1S,9S)-1-amino-9-ethyl-5-fluoro-1,2,3,9,12,15-hexahydro-9-hydroxy-4-methyl-10H, 13H-benzo[de] pyrano[3′,4′:6,7] indolizino[1,2-b]quinolin-10,13-dione, (also expressed as chemical name: (1S,9S)-1-amino-9-ethyl-5-fluoro-2,3-dihydro-9-hydroxy-4-methyl-1H, 12H-benzo[de] pyrano[3′, 4′: 6,7] indolizino[1,2-b]quinolin-10,13 (9H, 15H)-dione)), which is a topoisomerase I inhibitor, as a component. Exatecan is a camptothecin derivative having an antitumor effect, represented by the following formula:

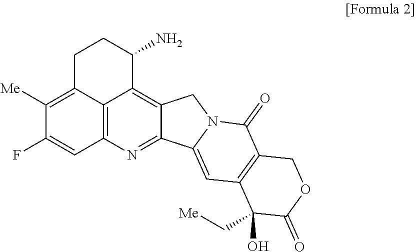 [SMILES: CC[C@@]1(O)C(=O)OCc2c1cc1n(c2=O)Cc2c-1nc1cc(F)c(C)c3c1c2[C@@H](N)CC3]

The antibody-drug conjugate used in embodiments of the present invention can also be represented by the following formula:

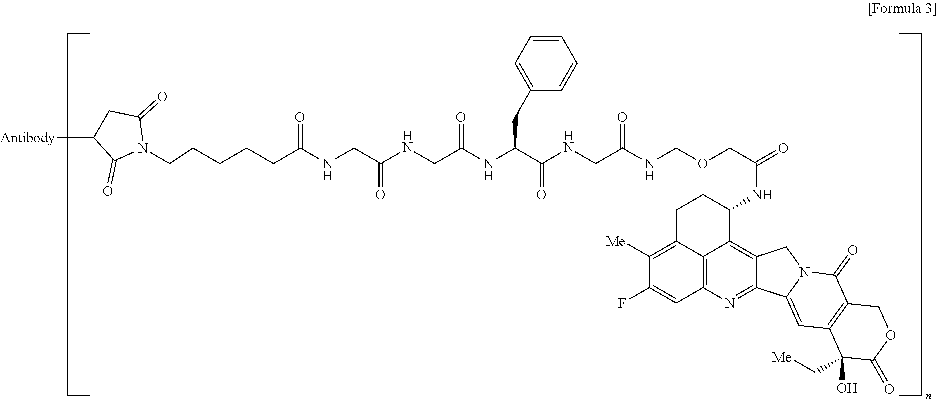

wherein, the drug-linker is conjugated to the antibody specific for the tumor antigen via a thioether bond. The meaning of n is the same as that of what is called the average number of conjugated drug molecules (DAR; Drug-to-Antibody Ratio), and indicates the average number of units of the drug-linker conjugated per antibody molecule.

After migrating into cancer cells, the antibody-drug conjugate used in embodiments of the present invention is cleaved at the linker portion to release the compound represented by the following formula (hereinafter referred to as compound (A)):

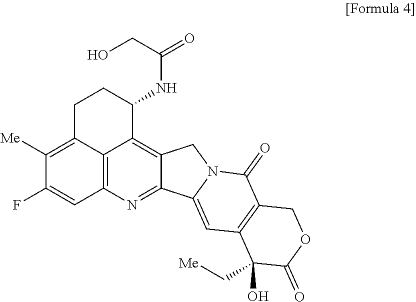 [SMILES: CC[C@@]1(O)C(=O)OCc2c1cc1n(c2=O)Cc2c-1nc1cc(F)c(C)c3c1c2[C@@H](NC(=O)CO)CC3]

The aforementioned compound is inferred to be the original source of at least some of the antitumor activity of the antibody-drug conjugate used in such embodiments of the present invention, and has been confirmed to have a topoisomerase I inhibitory effect (Ogitani Y. et al., Clinical Cancer Research, 2016 Oct. 15; 22 (20): 5097-5108, Epub 2016 Mar. 29.)

Topoisomerase I is an enzyme, which cleaves a DNA single-strand and rejoins cleaved fragments, is involved in the conversion of a DNA higher-order structure and synthesis of DNA. Thus, a drug having a topoisomerase I inhibitory effect inhibits DNA synthesis to terminate cell division during the S phase (DNA synthesis phase) in the cell cycle and induces apoptosis (cell death) to suppress cancer-cell proliferation.

The antibody-drug conjugate used in embodiments of the present invention is also known to have a bystander effect (Ogitani Y. et al., Cancer Science (2016) 107, 1039-1046).

The bystander effect is exerted through a process such that the antibody-drug conjugate used in such embodiments of the present invention is internalized in cancer cells expressing a target, and the aforementioned compound is released then exerts an antitumor effect also on cancer cells which are present therearound and not expressing the target.

The bystander effect is also exerted as an excellent antitumor effect when the antibody-drug conjugate according to such embodiments of the present invention is used in combination with a CD47 inhibitor.

### 4. Production of the Antibody-Drug Conjugate

A drug-linker intermediate for use in the production of the antibody-drug conjugate according to the present invention is represented by the following formula.

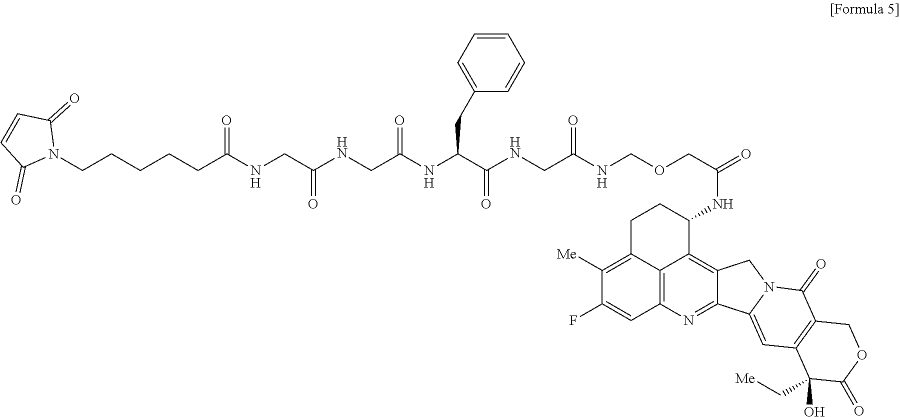 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

The drug-linker intermediate can be expressed as the chemical name N-[6-(2,5-dioxo-2,5-dihydro-1H-pyrrol-1-yl) hexanoyl]glycylglycyl-L-phenylalanyl-N-[(2-{[(1S,9S)-9-ethyl-5-fluoro-9-hydroxy-4-methyl-10,13-dioxo-2,3,9,10,13,15-hexahydro-1H, 12H-benzo[de] pyrano[3′,4′:6,7] indolizino[1,2-b]quinolin-1-yl] amino}-2-oxoethoxy)methyl]glycinamide, and can be produced with reference to descriptions of International Publication No. WO 2014/057687, International Publication No. WO 2015/098099, International Publication No. WO 2015/115091, International Publication No. WO 2015/155998, and International Publication No. WO 2019/044947.

The antibody-drug conjugate used in embodiments of the present invention can be produced by reacting the above-described drug-linker intermediate and an antibody having a thiol group (alternatively referred to as a sulfhydryl group), the antibody being specific for a tumor antigen.

The antibody having a sulfhydryl group can be obtained by a method well-known in the art (Hermanson, G. T, Bioconjugate Techniques, pp. 56-136, pp. 456-493, Academic Press (1996)). For example, by using 0.3 to 3 molar equivalents of a reducing agent such as tris(2-carboxyethyl) phosphine hydrochloride (TCEP) per interchain disulfide within the antibody and reacting with the antibody in a buffer solution containing a chelating agent such as ethylenediamine tetraacetic acid (EDTA), an antibody having a sulfhydryl group with partially or completely reduced interchain disulfides within the antibody can be obtained.

Further, by using 2 to 20 molar equivalents of the drug-linker intermediate per the antibody having a sulfhydryl group, an antibody-drug conjugate in which 2 to 8 drug molecules are conjugated per antibody molecule can be produced.

The average number of conjugated drug molecules per antibody molecule of the antibody-drug conjugate produced can be determined, for example, by a method of calculation based on measurement of UV absorbance for the antibody-drug conjugate and the conjugation precursor thereof at two wavelengths of 280 nm and 370 nm (UV method), or a method of calculation based on quantification through HPLC measurement for fragments obtained by treating the antibody-drug conjugate with a reducing agent (HPLC method).

Conjugation between the antibody and the drug-linker intermediate and calculation of the average number of conjugated drug molecules per antibody molecule of the antibody-drug conjugate can be performed with reference to descriptions in International Publication No. WO 2014/057687, International Publication No. WO 2015/098099, International Publication No. WO 2015/115091, International Publication No. WO 2015/155998, International Publication No. WO 2018/135501, and International Publication No. WO 2018/212136, and so on.

In the present invention, “anti-HER2 antibody-drug conjugate” represents an antibody-drug conjugate in which the antibody in the antibody-drug conjugate according to the invention is an anti-HER2 antibody.

The average number of units of the drug-linker conjugated per antibody molecule in the anti-HER2 antibody-drug conjugate is preferably 2 to 8, more preferably 3 to 8, even more preferably 7 to 8, even more preferably 7.5 to 8, and even more preferably about 8.

The anti-HER2 antibody-drug conjugate can be produced with reference to descriptions in International Publication No. WO 2015/115091 and so on.

In the present invention, the term “anti-HER3 antibody-drug conjugate” refers to an antibody-drug conjugate such that the antibody in an antibody-drug conjugate according to the invention is an anti-HER3 antibody.

The average number of units of the drug-linker conjugated per antibody molecule in the anti-HER3 antibody-drug conjugate is preferably 2 to 8, more preferably 3 to 8, even more preferably 7 to 8, even more preferably 7.5 to 8, and even more preferably about 8.

The anti-HER3 antibody-drug conjugate can be produced with reference to descriptions in International Publication No. WO 2015/155998 and so on.

In the present invention, the term “anti-TROP2 antibody-drug conjugate” refers to an antibody-drug conjugate such that the antibody in the antibody-drug conjugate according to the invention is an anti-TROP2 antibody.

The average number of units of the drug-linker conjugated per antibody molecule in the anti-TROP2 antibody-drug conjugate is preferably 2 to 8, more preferably 3 to 5, even more preferably 3.5 to 4.5, and even more preferably about 4.

The anti-TROP2 antibody-drug conjugate can be produced with reference to descriptions in International Publication No. WO 2015/098099 and so on.

In the present invention, the term “anti-B7-H3 antibody-drug conjugate” refers to an antibody-drug conjugate such that the antibody in the antibody-drug conjugate according to the invention is an anti-B7-H3 antibody.

The average number of units of the drug-linker conjugated per antibody molecule in the anti-B7-H3 antibody-drug conjugate is preferably 2 to 8, more preferably 3 to 5, even more preferably 3.5 to 4.5, and even more preferably about 4.

The anti-B7-H3 antibody-drug conjugate used in the present invention can be produced with reference to descriptions in International Publication No. WO 2014/057687 and so on.

In the present invention, the term “anti-GPR20 antibody-drug conjugate” refers to an antibody-drug conjugate such that the antibody in the antibody-drug conjugate according to the invention is an anti-GPR20 antibody.

The average number of units of the drug-linker conjugated per antibody molecule in the anti-GPR20 antibody-drug conjugate is preferably 2 to 8, more preferably 3 to 8, even more preferably 7 to 8, even more preferably 7.5 to 8, and even more preferably about 8.

The anti-GPR20 antibody-drug conjugate can be produced with reference to descriptions in International Publication No. WO 2018/135501 and so on.

In the present invention, the term “anti-CDH6 antibody-drug conjugate” refers to an antibody-drug conjugate such that the antibody in the antibody-drug conjugate according to the invention is an anti-CDH6 antibody.

The average number of units of the drug-linker conjugated per antibody molecule in the anti-CDH6 antibody-drug conjugate is preferably 2 to 8, more preferably 3 to 8, even more preferably 7 to 8, even more preferably 7.5 to 8, and even more preferably about 8.

The anti-CDH6 antibody-drug conjugate can be produced with reference to descriptions in International Publication No. WO 2018/212136 and so on.

### 5. Relationship Between Immunogenic Cell Death and CD47 Inhibitor

Immunogenic cell death (ICD) is cell death characterized by massive release of intracellular molecules such as ATP and HMGB1 (High-mobility group box1 protein) and exposure of Calreticulin (CRT) on the cell surface. These are Danger signals, which activate immune cells. It is reported that ATP is responsible for recruiting and activating dendritic cells (DC) and macrophages; HMGB1 is responsible for enhancing the production of inflammatory cytokines such as Type I IFN; and CRT serves as “eat-me-signal” and enhances antigen uptake from dead cells (Nature Reviews Immunology. 2017, 17, 97-111). In short, ICD of cancer cells occurs, and immunity (antitumor immunity) against the cancer cells can be induced. As the anti-cancer agent inducing ICD, e.g. an Anthracycline drug, Oxaliplatin and Cyclophosphamide are known; however, e.g., Docetaxel and Mitomycin C are not confirmed to induce ICD. The presence or absence of ICD effect can be evaluated not only in-vitro based on whether Danger-signal is detected or not by adding an agent but also in-vivo based on vaccination assay. In the latter one, cancer cells are treated with an agent and transplanted in an immunocompetent mouse. A week later, cancer cells not treated with the agent are transplanted to an opposite side. At this time, if immunological memory is already formed by the cancer cells, which caused ICD, engraftment and proliferation of the cancer cells transplanted are inhibited (Cancer Research, 2017, 77, 2686-2698).

For formation of immunological memory by ICD, it is important for myeloid cells such as dendritic cells and macrophages to take up a cancer antigen. At this time, it is presumed that a “Don't-eat-me signal” is transmitted between the myeloid cells and cancer cells by SIRPα-CD47. If a CD47 inhibitor is administered, the signal transmission can be inhibited and phagocytosis is enhanced. As a result, the uptake of the cancer antigen can be enhanced. The cancer antigen taken up by dendritic cells and/or macrophages is intracellularly processed into 8-30 mer peptide fragments, which are presented on MHC. MHC has two classes, class I and class II. An about 9-mer antigen peptide presented to MHC-class I activates CD8+ T cells; whereas an about 15-mer antigen peptide presented to MHC-class II activates CD4+T. Generally, a foreign antigen is processed in myeloid cells and presented to MHC-class II; however, part of DC subsets presents the foreign antigen to MHC-class I and activates CD8+T exerting cytotoxic activity to cancer cells; in short, the part of the DC subsets has cross-presentation ability.

The relationship of an antibody-drug conjugate, in which a drug having a cytocidal activity is integrated therein, and ICD induction, has been reported in the past in the cases of antibody-drug conjugates having Tubulysin, Pyrrolobenzodiazepine (PBD) and MMAE integrated therein (Cancer Research, 2017, 77, 2686-2698 or ONCOIMMUNOLOGY, 2019, 8 (4), e1565859). With respect to an antibody-drug conjugate used in the present invention containing a TpoI inhibitor, compound (A), as a payload, it has been only reported that HMGB1 is released from cells treated with compound (A) and a limited antitumor effect is exerted by vaccination of the treated cells (Clin. Invest. 2020:130 (1): 374-388). This time, the following is found: 1) compound (A) induces release of not only HMGB1 but also other Danger signals from dying cancer cells; and 2) compound (A) activates immune cells present under the microenvironment of cancer and enhances a cancer antigen-specific T cell population to induce antitumor immunity.

The mechanism of action of the present invention will now be explained. Firstly, antibodies specific for a tumour antigen bind to cancer cells. The antibodies consequently induce ADCP via nearby immune cells. Secondly, the CD47 inhibitor enhances ADCP activity by cancelling the “don't-eat-me signal” that is presented by the cancer cells. Thirdly, in embodiments where the antibodies specific for a tumour antigen are part of ADCs, the antibodies deliver the drug to the cancer cells, the drug payload thereby inducing immunogenic cell death of the cancer cells (to the extent that the cells are not also phagocytosed), which releases inflammatory molecules such as ATP and HMGB1. These molecules can activate immune cells. As such, the immunogenic cell death due to the drug payload and the enhanced ADCP by the antibody portion of the antibody-drug conjugates in the presence of CD47 inhibitor contributes to anti-tumor immunity.

### 6. Treatment, Dosage and Combinations

In accordance with the therapeutic method of the present invention, a CD47 inhibitor and an antibody specific for a tumor antigen are administered in combination.

In some embodiments of the therapeutic method of the present invention, the CD47 inhibitor and the antibody specific for the tumor antigen are separately contained as active components in different formulations. In some embodiments, the CD47 inhibitor and the antibody specific for the tumor antigen are administered simultaneously, whereas in other embodiments they are administered at different times (i.e. sequentially). In still further embodiments, the CD47 inhibitor and the antibody specific for the tumor antigen are contained as active components in a single formulation and are administered together.

The pharmaceutical composition or therapeutic method of the present invention can be used for treating cancer, preferably for treating at least one disease selected from the group consisting of breast cancer, gastric cancer (also called to as gastric adenocarcinoma), colorectal cancer (also called colon and rectal cancer, and including colon cancer and rectal cancer), lung cancer (including small cell lung cancer and non-small cell lung cancer), esophageal cancer, head-and-neck cancer (including salivary gland cancer and pharyngeal cancer), esophagogastric junction adenocarcinoma, biliary tract cancer (including bile duct cancer), gallbladder cancer, Paget's disease, pancreatic cancer, ovarian cancer, uterine carcinosarcoma, urothelial cancer, prostate cancer, bladder cancer, gastrointestinal stromal tumor, uterine cervix cancer, squamous cell carcinoma, peritoneal cancer, liver cancer, hepatocellular cancer, endometrial cancer, kidney cancer, vulval cancer, thyroid cancer, thymus cancer, penis cancer, leukemia, lymphoma, malignant lymphoma, plasmacytoma, myeloma, myelodysplastic syndrome, brain tumor, glioma, glioblastoma multiforme, osteosarcoma, and melanoma; more preferably for treating at least one cancer selected from the group consisting of breast cancer, gastric cancer, colorectal cancer, lung cancer, esophageal cancer, salivary gland cancer, esophagogastric junction adenocarcinoma, biliary tract cancer, Paget's disease, pancreatic cancer, ovarian cancer, bladder cancer, prostate cancer, and uterine carcinosarcoma; and even more preferably for treating at least one cancer selected from the group consisting of breast cancer, gastric cancer, lung cancer, and ovarian cancer.

In preferred embodiments, the specificity of the antibody specific for the tumor antigen is determined by examining the type of cancer and/or tumor marker present. For example, if HER2 expression is found in a cancer, an anti-HER2 antibody can preferably be used; if HER3 expression is found in a cancer, an anti-HER3 antibody can preferably be used; if TROP2 expression is found in a cancer, an anti-TROP2 antibody can preferably be used; if B7-H3 expression is found in a cancer, an anti-B7-H3 antibody can preferably be used; if GPR20 expression is found in a cancer, an anti-GPR20 antibody can preferably be used; if CDH6 expression is found in a cancer, an anti-CDH6 antibody can preferably be used.

The presence or absence of HER2, HER3, TROP2, B7-H3, GPR20, and CDH6, and other tumor markers can be checked by, for example, collecting tumor tissue from a cancer patient, and subjecting the formalin-fixed paraffin-embedded specimen (FFPE) to an examination at a gene product (protein) level such as an immunohistochemistry (IHC) method, a flow cytometry, a western blot method, or examination at a gene translation level such as an in situ hybridization method (ISH), a quantitative PCR method (q-PCR), a microarray analysis, alternatively, can also be checked by collecting cell-free blood circulating tumor DNA (ctDNA) from a cancer patient and subjecting to an examination which uses a method such as next generation sequencing (NGS).

The pharmaceutical composition and therapeutic method of the present invention can preferably be used for mammals, and can more preferably be used for humans.

An antitumor effect of the pharmaceutical composition and therapeutic method of the present invention can be confirmed by, for example, generating a model in which cancer cells are transplanted to a test animal, and measuring the reduction of tumor volume, life-prolonging effect due to treating the pharmaceutical composition and therapeutic method of the present invention. Furthermore, comparison to antitumor effects in single administrations of each of the CD47 inhibitor and antibody specific for a tumor antigen used in the present invention can provide confirmation of a combined effect of the CD47 inhibitor and antibody specific for a tumor antigen used in the present invention.

In addition, an antitumor effect of the pharmaceutical composition and therapeutic method of the present invention can be confirmed, in a clinical study, with Response Evaluation Criteria in Solid Tumors (RECIST) evaluation method, WHO's evaluation method, Macdonald's evaluation method, measurement of body weight, and other methods; and can be determined by indicators such as Complete response (CR), Partial response (PR), Progressive disease (PD), Objective response rate (ORR), Duration of response (DoR), Progression-free survival (PFS), and Overall survival (OS).

The foregoing methods can provide the confirmation of superiority in terms of the antitumor effect of the pharmaceutical composition and therapeutic method of the present invention to existing pharmaceutical compositions and therapeutic methods for cancer therapy.

The pharmaceutical composition and therapeutic method of the present invention can retard cancer cell growth, suppress its proliferation, and further can disrupt cancer cells. These effects can allow cancer patients to be free from symptoms caused by cancer or achieve improvement in QOL of a cancer patients, and attain a therapeutic effect by sustaining the lives of the cancer patients. Even if the pharmaceutical composition and therapeutic method of the present invention do not accomplish killing cancer cells, they can achieve higher QOL of cancer patients while achieving longer-term survival, by inhibiting, controlling or preventing the growth of cancer cells.

The pharmaceutical composition of the present invention can exert a therapeutic effect by application as systemic therapy to patients, and additionally, by local application to cancer tissues.

The pharmaceutical composition of the present invention may be administered, containing at least one pharmaceutically suitable ingredient. The pharmaceutically suitable ingredient can be suitably selected and applied from formulation additives or the like that are generally used in the art, in view of the dosage, administration concentration or the like of the CD47 inhibitor and antibody specific for a tumor antigen used in the present invention. For example, the antibody specific for a tumor antigen used in the present invention may be administered as a pharmaceutical composition containing a buffer such as a histidine buffer, an excipient such as sucrose or trehalose, and a surfactant such as polysorbate 80 or 20. The pharmaceutical composition containing the antibody specific for a tumor antigen used in the present invention can preferably be used as an injection, can more preferably be used as an aqueous injection or a lyophilized injection, and can even more preferably be used as a lyophilized injection.

In the case that the pharmaceutical composition containing the antibody specific for a tumor antigen used in the present invention is an aqueous injection, it can preferably be diluted with a suitable diluent and then given as an intravenous infusion. For the diluent, a dextrose solution, physiological saline, and the like, can be exemplified, and a dextrose solution can be exemplified preferably, and a 5% dextrose solution can be exemplified more preferably.

In the case that the pharmaceutical composition containing the antibody specific for a tumor antigen used in the present invention is a lyophilized injection, it can preferably be dissolved in water for injection, subsequently a required amount can be diluted with a suitable diluent and then given as an intravenous infusion. For the diluent, a dextrose solution, physiological saline, and the like, can be exemplified, and a dextrose solution can be exemplified preferably, and a 5% dextrose solution can be exemplified more preferably.

Examples of the administration route which may be used to administer the pharmaceutical composition of present invention include intravenous, intradermal, subcutaneous, intramuscular, and intraperitoneal routes; and preferably include an intravenous route.

The antibody-drug conjugate specific for a tumor antigen used in the present invention can be administered to a human once with intervals of 1 to 180 days, and can preferably be administered once a week, once every 2 weeks, once every 3 weeks, or once every 4 weeks, and can even more preferably be administered, once every 3 weeks. Also, the antibody-drug conjugate specific for a tumor antigen used in the present invention can be administered at a dose of about 0.001 to 100 mg/kg, and can preferably be administered at a dose of 0.8 to 12.4 mg/kg. In the case that the antibody-drug conjugate specific for a tumor antigen in the present invention is an anti-HER2 antibody-drug conjugate, it can preferably be administered once every 3 weeks at a dose of 0.8 mg/kg, 1.6 mg/kg, 3.2 mg/kg, 5.4 mg/kg, 6.4 mg/kg, 7.4 mg/kg, or 8 mg/kg. In the case that the antibody-drug conjugate specific for a tumor antigen used in the present invention is an anti-HER3 antibody-drug conjugate, it can preferably be administered once every 3 weeks at a dose of 1.6 mg/kg, 3.2 mg/kg, 4.8 mg/kg, 5.6 mg/kg, 6.4 mg/kg, 8.0 mg/kg, 9.6 mg/kg, or 12.8 mg/kg. In the case that the antibody-drug conjugate specific for a tumor antigen used in the present invention is an anti-TROP2 antibody-drug conjugate, it can preferably be administered once every 3 weeks at a dose of 0.27 mg/kg, 0.5 mg/kg, 1.0 mg/kg, 2.0 mg/kg, 4.0 mg/kg, 6.0 mg/kg, 8.0 mg/kg, or 10.0 mg/kg. The CD47 inhibitor according to the present invention can be administered to a human once with intervals of 1 to 180 days, and can preferably be administered once a week, once every 2 weeks, once every 3 weeks, or once every 4 weeks. Also, the CD47 inhibitor according to the present invention can be administered at a dose of about 0.001 to 100 mg/kg per dose.

The pharmaceutical composition and therapeutic method of the present invention may further contain a cancer therapeutic agent other than the CD47 inhibitor and the antibody specific for a tumor antigen according to the present invention. The pharmaceutical composition and therapeutic method of the present invention can also be administered in combination with another cancer therapeutic agent, thereby enhancing an antitumor effect. Another cancer therapeutic agent to be used for such purpose may be administered to a subject simultaneously, separately, or sequentially with the pharmaceutical composition of the present invention, or may be administered with varying dosage intervals. Such cancer therapeutic agents are not limited as long as they are agents having antitumor activity, and can be exemplified by at least one selected from the group consisting of irinotecan (CPT-11), cisplatin, carboplatin, oxaliplatin, fluorouracil (5-FU), gemcitabine, capecitabine, doxorubicin, epirubicin, cyclophosphamide, mitomycin C, tegafur-gimeracil-oteracil combination, panitumumab, bevacizumab, ramucirumab, regorafenib, trifluridine-tipiracil combination, gefitinib, erlotinib, afatinib, methotrexate, pemetrexed, tamoxifen, toremifene, fulvestrant, leuprorelin, goserelin, letrozole, anastrozole, progesterone formulation, and lapatinib.

In the pharmaceutical composition and therapeutic method of the present invention, an immune checkpoint inhibitor and an antibody-drug specifically binding to a cancer antigen and having ADCC and/or ADCP activity may be further contained as the cancer therapeutic agent to be used in combination, other than the CD47 inhibitor and the antibody specific for a tumor antigen according to the present invention. As the immune checkpoint inhibitor, an inhibitor of binding between PD-1 and its ligand, PD-L1, or a CTLA4 inhibitor is exemplified. Specific examples thereof include an anti-PD-1 antibody (nivolumab, pembrolizumab, cemiplimab, spartalizumab, PDR-001, or BI 754091), an anti-PD-L1 antibody (atezolizumab, avelumab, or durbarumab), and an anti-CTLA4 antibody (ipilimumab or tremelimumab). Examples of the antibody-drug specifically binding to a cancer antigen and having ADCC and/or ADCP activity include an anti-CD20 antibody (rituximab), an anti-HER2 antibody (trastuzumab or pertuzumab), an anti-EGFR antibody (cetuximab), and an anti-CD52 antibody (alemutuzumab).

ADCC refers to a cell-mediated reaction in which non-specific cytotoxic cells (for example, NK cells, neutrophils and macrophages) expressing an Fcγ receptor recognize an antibody bound to a target cell, and then, causes lysis of the target cell. In NK cells, which are primary cells responsible for ADCC, FCYRIIC and FCYRIIIA are expressed. In monocytes, FCYRI, FCYRIIA, FCYRIIC and FCYRIIIA are expressed. On the other hand, ADCP refers to a cell-mediated reaction, in which phagocytes (for example, macrophages, neutrophils) expressing an Fc receptor recognizes an antibody bound to a target cell, and then, engulfs the target cell within the cells. In the monocytes, which are primary cells responsible for ADCP, FCYRI, FCYRIIA, FCYRIIC and FCYRIIIA are expressed.

The pharmaceutical composition and therapeutic method of the present invention can also be used in combination with radiotherapy. For example, a cancer patient receives radiotherapy before and/or after or simultaneously with receiving a therapy with the pharmaceutical composition of the present invention.

The pharmaceutical composition and therapeutic method of the present invention can also be used as an adjuvant chemotherapy in combination with surgical operation. The pharmaceutical composition of the present invention may be administered for the purpose of diminishing the size of tumor before surgical operation (referred to as pre-operative adjuvant chemotherapy or neoadjuvant therapy), or may be administered after surgical operation for the purpose of preventing the recurrence of any tumors (referred to as post-operative adjuvant chemotherapy or adjuvant therapy).

In some embodiments, the CD47 inhibitor and/or the antibody specific for a tumor antigen are formulated together with a pharmaceutically acceptable carrier, diluent, solubilizer, emulsifier, preservative, auxiliary agent or others. The “pharmaceutically acceptable carrier” and others can be appropriately selected from a wide range in accordance with the type of target disease and the dosage form of a drug. A method for administering the CD47 inhibitor and/or the antibody specific for a tumor antigen according to the present invention can be appropriately selected, for example, administration by injection can be selected. Examples of the injection that can be employed include local injection, intraperitoneal injection, selective intravenous injection, intravenous injection, subcutaneous injection and organ-perfusate injection. The injection solution can be formulated by using a carrier consisting of a salt solution, a glucose solution, or a mixture of a salt solution and a glucose solution, and any one of the buffers. Alternatively, the injection solution may be prepared by formulating a powder preparation and mixing the powder preparation with the aforementioned liquid carrier, when used.

Other administration e appropriately selected together with the development of formulations. For example, for oral administration, oral liquids, powders, pills, capsules and tablets can be applied. In the case of an oral liquid, an oral liquid preparation such as a suspension and a syrup can be produced by using water; a sugar such as sucrose, sorbitol and fructose; a glycol such as polyethylene glycol; an oil such as sesame oil and soybean oil; a preservative such as alkyl parahydroxybenzoate; and a flavor such as strawberry flavor and peppermint flavor. Powders, pills, capsules and tablets can be formulated by using, e.g., an excipient such as lactose, glucose, sucrose and mannitol; a disintegrant such as starch and soda alginate; a lubricant such as magnesium stearate and talc; a binder such as polyvinyl alcohol, hydroxypropyl cellulose and gelatin; a surfactant such as a fatty acid ester; and a plasticizer such as glycerin. Tablets and capsules are easily administered. In this respect, they are preferable unit dosage forms of the composition of the invention. In producing tablets and capsules, a solid production carrier is used.

### EXAMPLES

The present invention is specifically described in view of the examples shown below. However, the present invention is not limited to these. Further, it is by no means to be interpreted in a limited way.

### Production Example 1: Production of the Antibody-Drug Conjugate (1)

In accordance with a production method described in International Publication No. WO 2015/115091 with use of a humanized anti-HER2 antibody (an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2, hereinafter will be “referred to as humanized anti-HER2 antibody (1)”), an antibody-drug conjugate in which a drug-linker represented by the following formula:

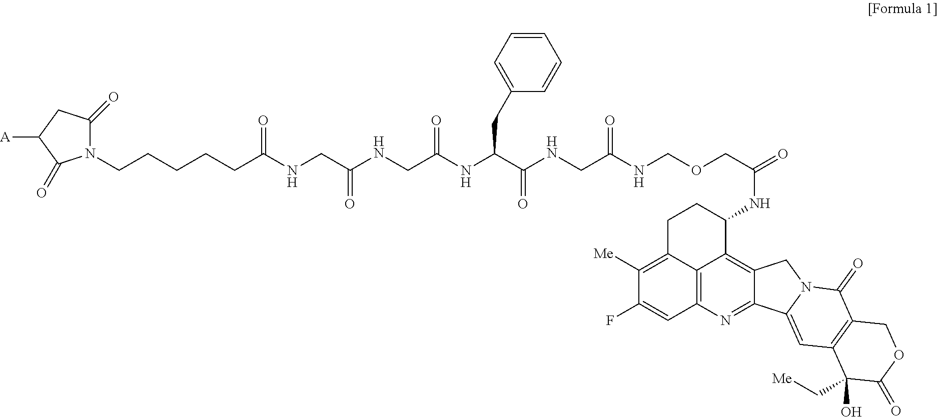

wherein A represents the connecting position to an antibody, is conjugated to an anti-HER2 antibody via a thioether bond (hereinafter referred to as “antibody-drug conjugate (1)”) was produced. The DAR of the antibody-drug conjugate (1) is 7.7 or 7.8.

### Production Example 2: Production of the Antibody-Drug Conjugate (2)

In accordance with a production method described in Publication Nos. WO 2015/098099 and International 2017/002776 with use of a humanized anti-TROP2 antibody (an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6, hereinafter will be referred to as humanized anti-TROP2 antibody (1)), an antibody-drug conjugate in which a drug-linker represented by the following formula:

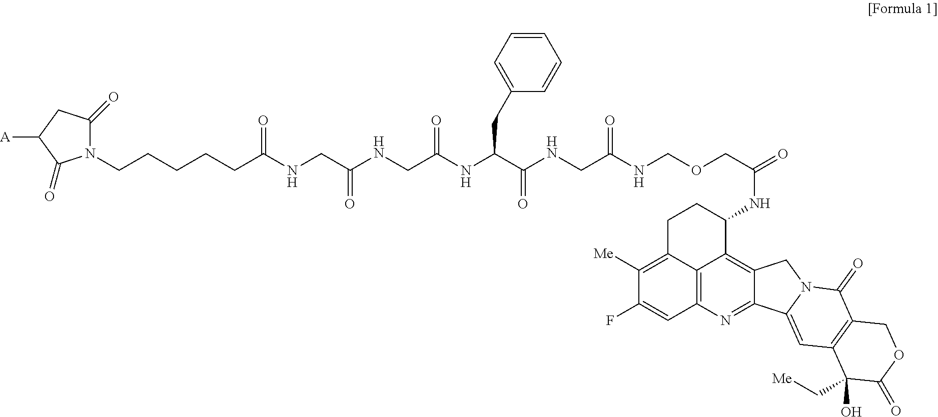

wherein A represents the connecting position to an antibody, is conjugated to an anti-TROP2 antibody via a thioether bond (hereinafter referred to as “antibody-drug conjugate (2)”) was produced. Although DAR of the antibody-drug conjugate (2) can be controlled within the range of from 0 to 8, an antibody-drug conjugate having an average number of conjugated drugs of 3.5 to 4.5 was produced herein.

### Production Example 3: Production of the Antibody-Drug Conjugate (3)

In accordance with a production method described in International Publication No. WO 2014/057867 and 2017/002776 with use of a humanized anti-B7-H3 antibody (an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8, hereinafter will be referred to as humanized anti-B7-H3 antibody (3)), an antibody-drug conjugate in which a drug-linker represented by the following formula:

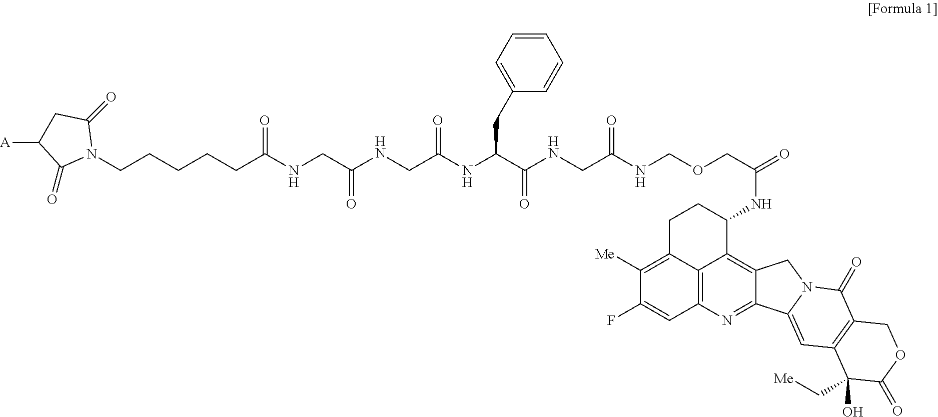

wherein A represents the connecting position to an antibody, is conjugated to an anti-B7-H3 antibody via a thioether bond (hereinafter referred to as “antibody-drug conjugate (3)”) was produced. Although DAR of the antibody-drug conjugate (3) can be controlled within the range of from 0 to 8, an antibody-drug conjugate having an average number of conjugated drugs of 3.5 to 4.5 was produced herein.

### Example 1: ADCP Activity of Antibody-Drug Conjugate of Compound (a) and the Anti-CD47 Blocker to HER2-Positive and TROP2-Positive Cancer Cell Lines 1-1 Preparation of target cells

Human gastric cancer cell line AGS (CD47 and TROP2 positive) and human breast cancer cell line JIMT-1 (CD47 and HER2 positive) were collected, washed twice with PBS and resuspended in PBS. The number of living cells were counted in accordance with the trypan blue exclusion test. A CellTrace Violet (CTV, Thermo Fisher Scientific) solution was added in a volume of 1 UL per 1×10⁶ cells/mL. The mixture was allowed to stand still at room temperature for 10 minutes in the dark. After the incubation, the cells were washed twice with 10 mL of 10% FBS containing RPMI1640 culture medium (R10). Finally, the cell suspension was made so as to be 1×10⁶ cells/mL and used as target cells.

### 1-2 Preparation of Effector Cells

On Day 0, to SepMate-50 tubes (STEMCELL Technologies), Ficoll-Paque PLUS (GE Healthcare) was dispensed in an amount of 15 mL/tube. The whole blood diluted 2-fold with 2% FBS containing PBS was overlaid in an amount of 33-34 mL/tube. Centrifugation was performed at room temperature at 1200 g for 10 minutes. The supernatant containing PBMCs was collected in fresh 50 mL tubes by decantation, added with 10 mL of 2% FBS containing PBS, and centrifuged at room temperature at 300 g for 5-8 minutes. After the supernatant was removed, the PBMCs were washed twice with 10 mL of 2% FBS containing PBS. The number of live PBMCs was counted and the PBMCs were suspended in RoboSep buffer (STEMCELL Technologies) at 5×10⁷ cells/mL. EasySep human Monocyte enrichment cocktail in Human monocyte Enrichment Kit without CD16 Depletion (STEMCELL Technologies) was added in an amount of 50 μL per 5×10⁷ PBMCs. After a reaction at 4° C. for 10 minutes, EasySep Magnetic Particles (STEMCELL Technologies) were added in the PBMC suspension in an amount of 50 μL per 5×10⁷ PBMCs. After a reaction at 4° C. for 5 minutes, RoboSep buffer was added to the PBMCs up to 2.5 mL. The PBMCs were transferred to a 5 mL tube and placed in the magnetic field of EasySep Magnet (STEMCELL Technologies). Two minutes and 30 seconds later, the supernatant containing human peripheral blood monocytes was collected by decantation and centrifuged. To generate macrophages, the isolated monocytes were suspended in R10 containing 20 ng/ml M-CSF (PEPROTEC), seeded in a 225 cm² flask, and cultured at 37° C. and 5% CO₂ for 7 days. On day 7, the culture supernatant was removed, and R10 containing 20 ng/mL M-CSF was added. On day 11, the culture supernatant was removed, R10 containing 20 ng/mL M-CSF and 20 ng/mL IL-10 (PEPROTEC) was added. On day 13, the macrophages were washed once with PBS, added TrypLE Express (Thermo Fisher Scientific) and kept at 37° C. for 10-30 minutes. After harvesting, the macrophages were suspended in PBS at 1×10⁶ cells/mL. A CellTrace Far Red solution (Thermo Fisher Scientific) was added to the macrophages at 1 μL per 10⁶ cells/mL. After the incubation at room temperature for 10 minutes in the dark, the macrophages were washed twice with 10 mL of R10. The cells were suspended in R10 at 1×10⁶ cells/mL and used as effector cells.

### 1-3 Evaluation of ADCP Activity

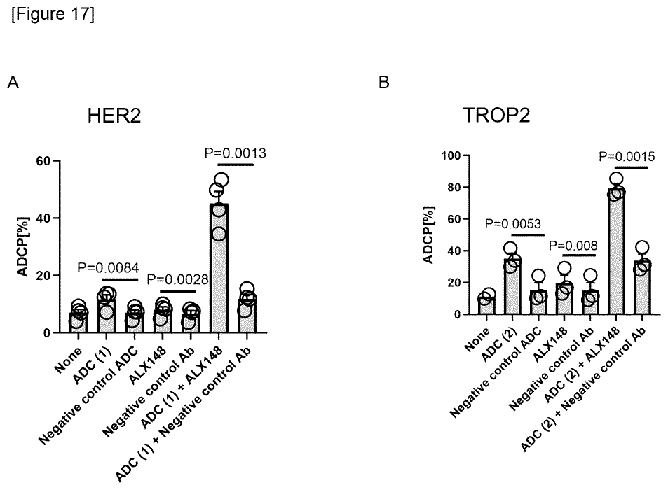

Firstly, to a U-bottom 96-well microplate, 100 μL of each of the indicated agents shown in FIG. 17 was added to the cells. Briefly, antibody-drug conjugate (1) or (2), or control antibody-drug conjugates diluted with R10 was added at a final concentration of 5 nM. ALX148 hIgG1 or the isotype-matched control Ab diluted with R10 was added at a final concentration of 50 nM. ALX148 hIgG1 was prepared with reference to US Publication US 2019/0169266 A1. The amino acid sequence of the ALX148 hIgG1 is set forth in SEQ ID NO: 15 in the sequence listing. Second, 50 μL of the effector cells (1×10⁶ cells/mL) prepared as above step 1-2 were added to the wells. Finally, 50 μL of the target cells (1×10⁶ cells/mL) prepared as above step 1-1 were added to the wells, and cells were mixed by gentle pipetting. The cells were incubated at 37° C. and 5% CO₂ for 4 hours. After centrifugation at 510 g for 3 minutes, the supernatant was removed. The cells were washed once with 200 μL of Hanks' Balanced Salt Solution containing 0.1% BSA. The cells were suspended in 50 μL of 1×BD Stabilizing Fixative (BD Biosciences) and kept at 4° C. in the dark until the flow cytometry analysis. The ADCP was assessed by BD LSRFortessa X-20 flow cytometer (BD Biosciences). The acquired data were analyzed by Flowjo (TreeStar). The cancer cells were determined as the CTV-positive (CTV+) cells. Within the cancer cell population, the phagocytosed cancer cells by macrophages were defined as CTV+ and Far Red positive (Far Red+), hereinafter referred to as CTV+ Far Red+ cells. ADCP (%) was calculated as follows:

$$ADCP ( \% ) = 100 \times [ ( CTV + FAR {Red}_{+} cell count ) \div ( {CTV}_{+} cell count ) ]$$

The data were analyzed in GraphPad Prism 9.1.0 (GraphPad Software), and the statistical analysis was performed in SAS System Release 9.2 (SAS Institute).

As shown in FIG. 17 the antibody-drug conjugate (1) or (2) showed ADCP activity to JIMT-1 and AGS cells, respectively, and showed higher ADCP activity in the presence of the ALX148_hIgG1 (FIGS. 17A, and 17B).

### Example 2: ADCP Activity of Antibody-Drug Conjugate of Compound (a) and the Anti-CD47 Blocker to B7-H3-Positive Cancer Cell Line

### 2-1 Preparation of Target Cells

Human lung cancer cell line NCI-H322 (CD47 and B7-H3 positive) was collected, washed twice PBS with and resuspended in PBS. The number of living cells were counted in accordance with the trypan blue exclusion test. A CellTrace Violet (CTV, Thermo Fisher Scientific) solution was added in a volume of 1 μL per 1×10⁶ cells/mL. The mixture was allowed to stand still at room temperature for 10 minutes in the dark. After the incubation, the cells were washed twice with 10 mL of 10% FBS containing RPMI1640 culture medium (R10). Finally, the cell suspension was made so as to be 1×10⁶ cells/mL and used as target cells.

### 2-2 Preparation of Effector Cells

On Day 0, to SepMate-50 tubes (STEMCELL Technologies), Ficoll-Paque PLUS (GE healthcare) was dispensed in an amount of 15 mL/tube. The whole blood diluted 2-fold with 2% FBS containing PBS was overlaid in an amount of 33-34 mL/tube. Centrifugation was performed at room temperature at 1200 g for 10 minutes. The supernatant containing PBMCs was collected in fresh 50 mL tubes by decantation, added with 10 mL of 2% FBS containing PBS, and centrifuged at room temperature at 300 g for 5 minutes. After the supernatant was removed, the PBMCs were washed twice with 10 mL of 2% FBS containing PBS. The number of live PBMCs was counted and the PBMCs were suspended in RoboSep buffer (STEMCELL Technologies) at 5×10⁷ cells/mL. EasySep human Monocyte enrichment cocktail in Human monocyte Enrichment Kit Without CD16 Depletion (STEMCELL Technologies) was added in an amount of 50 μL per 5×10⁷ PBMCs. After a reaction at 4° C. for 10 minutes, EasySep Magnetic Particles (STEMCELL Technologies) were added in the PBMC suspension in an amount of 50 μL per 5×10⁷ PBMCs. After a reaction at 4° C. for 5 minutes, RoboSep buffer was added to the PBMCs up to 2.5 mL. The PBMCs were transferred to a 5 mL tube and placed in the magnetic field of EasySep Magnet (STEMCELL Technologies). Two minutes and 30 seconds later, the supernatant containing human peripheral blood monocytes was collected by decantation and centrifuged. To generate macrophages, the isolated monocytes were suspended in R10 containing 20 ng/mL M-CSF (PEPROTEC), seeded in a 225 cm² flask, and cultured at 37° C. and 5% CO₂ for 7 days. On day 7, the culture supernatant was removed, and R10 containing 20 ng/mL M-CSF was added. On day 11, the culture supernatant was removed, R10 containing 20 ng/mL M-CSF and 20 ng/mL IL-10 (PEPROTEC) was added. On day 13, the macrophages were washed once with PBS, added TrypLE Express (Thermo Fisher Scientific) and kept at 37° C. for 10 minutes. After harvesting, the macrophages were suspended in PBS at 1×10⁶ cells/mL. A CellTrace Far Red solution (Thermo Fisher Scientific) was added to the macrophages at 1 μL per 10⁶ cells/mL. After the incubation at room temperature for 10 minutes in the dark, the macrophages were washed twice with 10 mL of R10. The cells were suspended in R10 at 1×10⁶ cells/mL and used as effector cells.

### 2-3 Evaluation of ADCP Activity

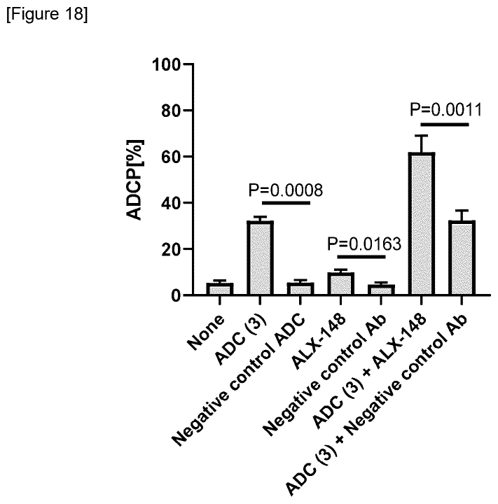

Firstly, 100 μL of each of the indicated agents shown in FIG. 18 was added to a U-bottom 96-well microplate. Briefly, antibody-drug conjugate (3), or control antibody-drug conjugates diluted with R10 was added at a final concentration of 5 nM. ALX148 hIgG1 or the isotype-matched control Ab diluted with R10 was added at a final concentration of 50 nM. ALX148 hIgG1 was prepared with reference to US Publication US 2019/0169266 A1. The amino acid sequence of the ALX148 hIgG1 is set forth in SEQ ID NO: 15 in the sequence listing. Second, 50 μL of the effector cells (1×10⁶ cells/mL) prepared as above step 2-2 were added to the wells. Finally, 50 μL of the target cells (1×10⁶ cells/mL) prepared as above step 2-1 were added to the wells, and cells were mixed by gentle pipetting. The cells were incubated at 37° C. and 5% CO₂ for 4 hours. After centrifugation at 510 g for 3 minutes, the supernatant was removed. The cells were washed once with 200 μL of Hanks' Balanced Salt Solution containing 0.1% BSA. The cells were suspended in 50 μL of 1×BD Stabilizing Fixative (BD Biosciences) and kept at 4° C. in the dark until the flow cytometry analysis. The ADCP was assessed by BD LSRFortessa X-20 flow cytometer (BD Biosciences). The acquired data were analyzed by Flowjo (TreeStar). The cancer cells were determined as the CTV positive (CTV+) cells. Within the cancer cell population, the phagocytosed cancer cells by macrophages were defined as CTV+ and Far Red positive (Far Red+), hereinafter referred to as CTV+ Far Red+ cells. ADCP (%) was calculated as follows:

$$ADCP ( \% ) = 100 \times [ ( {CTV}_{+} Far {Red}_{+} cell count ) ]$$

The data were analyzed in GraphPad Prism 9.1.0 (GraphPad Software), and the statistical analysis was performed in SAS System Release 9.2 (SAS Institute).

As shown in FIG. 18 the antibody-drug conjugate (3) showed ADCP activity against NCI-H322 cells and showed higher ADCP activity in the presence of the ALX148_hIgG1.

### Example 3: Antitumor Study (1)

Mouse: Female 6-week-old BALB/c mice (BALB/c AnNCr1Crlj) (The Jackson Laboratory Japan, INC.) were subjected to experiments.

Measurement and calculation formula: the long and short diameters of tumors were measured twice a week with an electronic digital caliper (Mitutoyo Corp), and the tumor volumes (mm³) were calculated as below:

$$Tumor volume ( {mm}^{3} ) = 0.5 \times long diameter ( mm ) \times {[ short diameter ( mm ) ]}^{2}$$

The antibody-drug conjugate (1) (Drug-to-Antibody Ratio: 7.8) was diluted with a buffer, and a dose of 10 mL/kg was intravenously administered to the tail vein. ALX148_mIgG1 was prepared with reference to US Publication US 2019/0169266 A1. The amino acid sequence of the ALX148 mIgG1 is set forth in SEQ ID NO: 16 in the sequence listing. ALX148_mIgG1 was diluted with saline, and a dose of 10 mL/kg was intraperitoneally administered.

A mouse colon cancer cell line CT26. WT (CRL-2638) was purchased from the American Type Culture Collection and transduced with a human HER2 gene using a retrovirus vector. The established CT26. WT-hHER2 were used for the experiment. On the day of inoculation of mice with CT26. WT-hHER2 cells, CT26. WT-hHER2 cells were harvested, suspended in PBS(−) at 3.0×10⁶ cells/head, and subcutaneously transplanted into the right abdominal region of each animal (Day 0). Six days later, the mice were randomly divided into groups based on the tumor volume (Day 6). The antibody-drug conjugate (1) was intravenously administered at a dose of 10 mg/kg on Days 6 and 13 (two times in total) to the tail vein. The ALX148 mIgG1 was intraperitoneally administered at a dose of 30 mg/kg on Days 7, 10 and 14 (three times in total). A combined administration group of the antibody-drug conjugate (1) and recombinant ALX148_mIgG1 was set up; and a vehicle administration group was set up as a control group. The number of mice per group was 8. Measurement of the tumor volume was continued until Day 27. Comparison among each of the single administration groups and the combined administration group was made in accordance with the Dunnett's multiple comparison. A P value less than 0.05 was defined as being statistically significant.

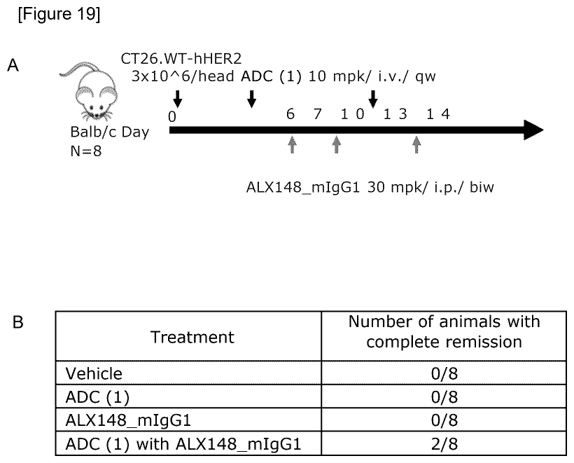

The results are shown in FIG. 19. FIG. 19A shows the treatment schedule of the experiment. FIG. 19B summarizes the number of animals with complete remission on Day 27. The combination of ADC (1) with ALX148 mIgG1 induced certain animals with complete remission (two out of eight mice), while the monotherapy of ADC (1) or ALX148_mIgG1 did not (zero out of eight mice).

### Example 4: Antitumor Study (2)

Mouse: Female 6-week-old B6J mice (C57BL/6J) (The Jackson Laboratory Japan, INC.) were subjected to experiments.

Measurement and calculation formula: the long and short diameters of tumors were measured twice a week with an electronic digital caliper (Mitutoyo Corp), and the tumor volumes [mm³] were calculated as below:

$$Tumor volume [ {mm}^{3} ] = 0.5 \times long diameter [ mm ] \times {[ short diameter [ mm ] ]}^{2} .$$

The antibody-drug conjugate (2) (Drug-to-Antibody Ratio: 3.9) was diluted with ABS, and a dose of 10 mL/kg was intravenously administered to the tail vein. Recombinant ALX148 mIgG1 was diluted with PBS, and intraperitoneally administered at a dose of 10 mL/kg.

A mouse colon cancer MC38 cell line was obtained from National Cancer Institute and transduced with a human TROP2 gene using a retrovirus vector. MC38-hTROP2 cells were suspended in saline, and 0.5×10⁶ cells were subcutaneously transplanted in the right axilla of each of the B6J mice (Day 0). Four days later, the mice were randomly divided into groups based on the tumor volume (Day 4). The antibody-drug conjugate (2) was intravenously administered at a dose of 1 mg/kg on Day 4 and 11 (two times in total) to the tail vein. ALX148_mIgG1 was intraperitoneally administered at a dose of 30 mg/kg on Day 5, 8 and 12 (three times in total). A combined administration group of the antibody-drug conjugate (2) and ALX148_mIgG1 and a vehicle administration group were set up. The number of mice per group was 12. Measurement of the tumor volume was continued until Day 18. Comparison among each of the single administration groups and the combined administration groups was made in accordance with the Dunnett's multiple comparison. A p-value less than 0.05 was defined as being statistically significant.

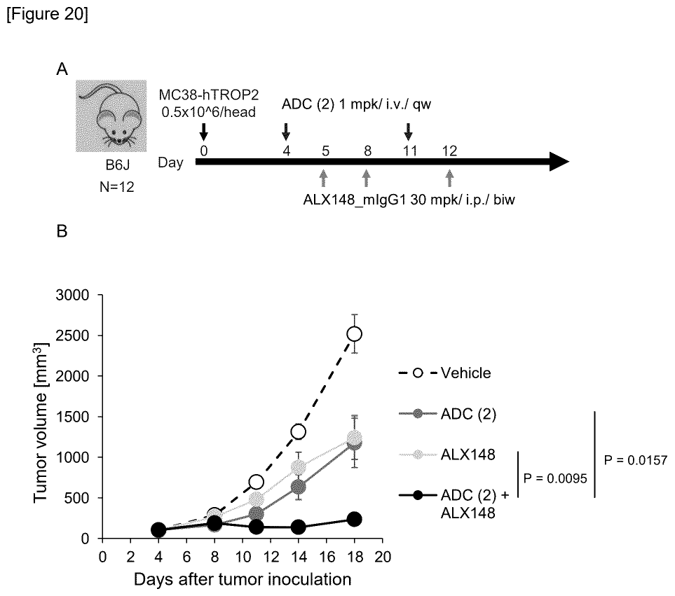

The results are shown in FIG. 20. FIG. 20A shows the treatment schedule of the experiment. FIG. 20B shows tumor growth of individual administration groups. The longitudinal axis represents tumor volume [mm³] and the abscissa axis represents the number of days after tumor inoculation. On Day 18, the combination of ADC (2) with ALX148_mIgG1 showed a superior anti-tumor effect over ADC (2) and ALX148_mIgG1 monotherapy, respectively (FIG. 20B).

From the above examples, it was found that the ADC according to the present invention shows an excellent antitumor effect if administered in combination with ALX148_mIgG1.

Since the mechanism of action of ALX148 is based on the blocking activity of CD47-SIRPα interaction, it is reasonable to expect that other CD47-targeting agents with the blocking activity would have the same combination effect.

1-80. (canceled)

81. A therapeutic method comprising administering a CD47 inhibitor and an antibody specific for a tumor antigen in combination to a subject in need of treatment, wherein the CD47 inhibitor is administered simultaneously, separately or sequentially with the antibody specific for a tumor antigen.

82. The method according to claim 81, wherein the CD47 inhibitor comprises SIRPα or a SIRPα derivative.

83. The method according to claim 82, wherein the SIRPα derivative comprises a polypeptide having a sequence with at least 80%, 90%, 95%, 99% or 100% sequence identity to the residues 1 to 149 of SEQ ID NO: 15 or 16.

84. The method according to claim 81, wherein the CD47 inhibitor is a fusion protein and further comprises an Fc region.

85. The method according to claim 84, wherein the CD47 inhibitor comprises a polypeptide having a sequence with at least 80%, 90%, 95%, 99% or 100% sequence identity to SEQ ID NO: 15 or 16.

86. The method according to claim 81, wherein the CD47 inhibitor is Evorpacept.

87. The method according to claim 82, wherein the CD47 inhibitor is selected from the group consisting of: TTI-621, TTI-622, DSP-107 and SL-172154.

88. The method according to claim 81 wherein the CD47 inhibitor comprises an antibody or an antigen-binding fragment thereof, wherein the antibody is specific for CD47.

89. The method according to claim 88, wherein the antibody specific for CD47 is selected from the group consisting of: Magrolimab, Lemsoparlimab, AO-176, SRF-231, IBI-188, IBI-322, IMC-002, MIL-95, TG-1801, ZL-1201, AK-117 (Ligufalimab) and IMM-0306.

90. The method according to claim 81, wherein the CD47 inhibitor comprises a small-molecule agent capable of binding to CD47.

91. The method according to claim 90, wherein the small molecule agent is selected from the group consisting of RRx-001 and IMM-01.

92. The method according to claim 81, wherein CD47 comprises a polypeptide sequence represented by SEQ ID NO: 13.

93. The method according to claim 81 comprising administering an antibody-drug conjugate, wherein the antibody specific for a tumor antigen is a part of the antibody-drug conjugate, and wherein the antibody-drug conjugate further comprises a linker and a drug, the antibody specific for a tumor antigen being connected to the drug via the linker, the linker and the drug forming a drug-linker.

94. The method according to claim 93, wherein the drug-linker is as represented by the following formula: 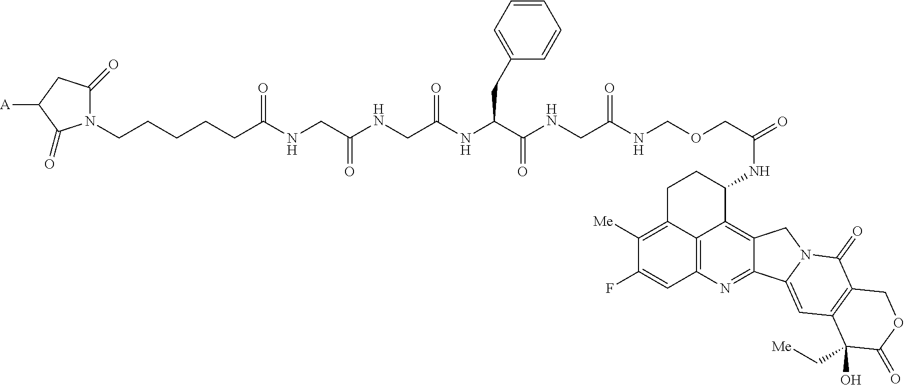
wherein A represents the connecting position to the antibody, and
wherein the drug-linker is conjugated to the antibody specific for a tumor antigen via a thioether bond.

95. The method according to claim 81, wherein the antibody specific for a tumor antigen is an anti-HER2 antibody, an anti-HER3 antibody, an anti-TROP2 antibody, an anti-B7-H3 antibody, an anti-GPR20 antibody, or an anti-CDH6 antibody.

96. The method according to claim 95, wherein the antibody specific for a tumor antigen is the anti-HER2 antibody.

97. The method according to claim 96, wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

98. The method according to claim 96, wherein the anti-HER2 antibody is an antibody comprising a heavy chain consisting of an amino acid sequence represented by SEQ ID NO: 1 and a light chain consisting of an amino acid sequence represented by SEQ ID NO: 2.

99. The method according to claim 96, wherein the anti-HER2 antibody is a part of an anti-HER2 antibody-drug conjugate further comprising a linker and a drug, wherein the anti-HER2 antibody is connected to the drug via the linker, the linker and the drug forming a drug-linker, wherein the drug-linker is as represented by the following formula: 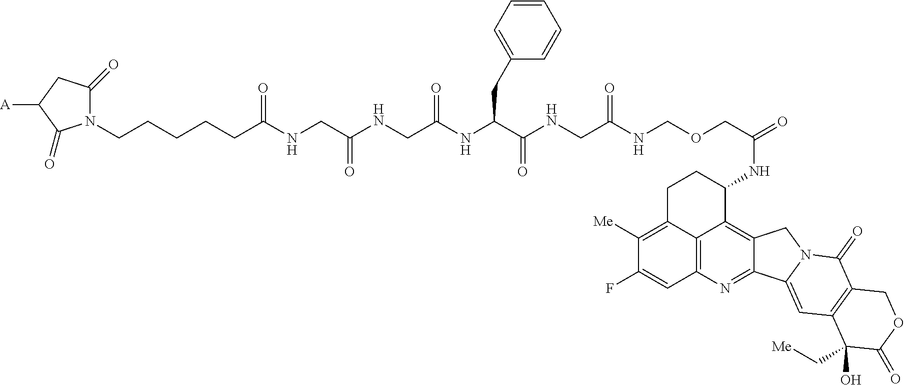
wherein A represents the connecting position to the antibody,
wherein the drug-linker is conjugated to the anti-HER2 antibody via a thioether bond; and
wherein the average number of units of the drug-linker conjugated per antibody molecule in the antibody-drug conjugate is in the range of from 7 to 8.

100. The method according to claim 95, wherein the antibody specific for a tumor antigen is an anti-HER3 antibody.

101. The method according to claim 100, wherein the anti-HER3 antibody is an antibody comprising a heavy chain consisting of an amino acid sequence represented by SEQ ID NO: 3 and a light chain consisting of an amino acid sequence represented by SEQ ID NO: 4.

102. The method according to claim 101, wherein the anti-HER3 antibody lacks a lysine residue at the carboxyl terminus of the heavy chain.

103. The method according to claim 100, wherein the anti-HER3 antibody is a part of an anti-HER3 antibody-drug conjugate further comprising a linker and a drug, wherein the anti-HER3 antibody is connected to the drug via the linker, the linker and the drug forming a drug-linker, wherein the drug-linker is as represented by the following formula: 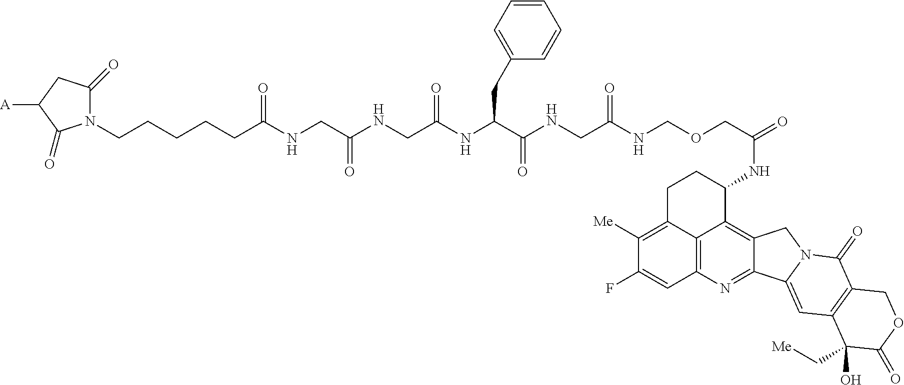
wherein A represents the connecting position to the antibody,
wherein the drug-linker is conjugated to the anti-HER3 antibody via a thioether bond; and
wherein the average number of units of the drug-linker conjugated per antibody molecule in the antibody-drug conjugate is in the range of from 7 to 8.

104. The method according to claim 95, wherein the antibody specific for a tumor antigen is an anti-TROP2 antibody.

105. The method according to claim 104, wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

106. The method according to claim 105, wherein the anti-TROP2 antibody lacks a lysine residue at the carboxyl terminus of the heavy chain.

107. The method according to claim 104, wherein the anti-HER2 antibody is a part of an anti-TROP2 antibody-drug conjugate further comprising a linker and a drug, wherein the anti-TROP2 antibody is connected to the drug via the linker, the linker and the drug forming a drug-linker, wherein the drug-linker is as represented by the following formula: 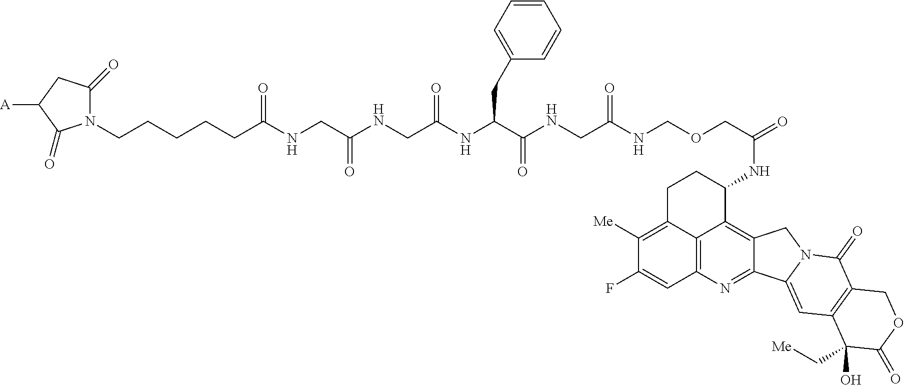
wherein A represents the connecting position to the antibody,
wherein the drug-linker is conjugated to the anti-TROP2 antibody via a thioether bond; and
wherein the average number of units of the drug-linker conjugated per antibody molecule in the antibody-drug conjugate is in the range of from 3.5 to 4.5.

108. The method according to claim 95, wherein the antibody specific for a tumor antigen is an anti-B7-H3 antibody.

109. The method according to claim 108, wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

110. The method according to claim 109, wherein the anti-B7-H3 antibody lacks a lysine residue at the carboxyl terminus of the heavy chain.

111. The method according to claim 108, wherein the anti-B7-H3 antibody is a part of an anti-B7-H3 antibody-drug conjugate further comprising a linker and a drug, wherein the anti-B7-H3 antibody is connected to the drug via the linker, the linker and the drug forming a drug-linker, wherein the drug-linker is as represented by the following formula: 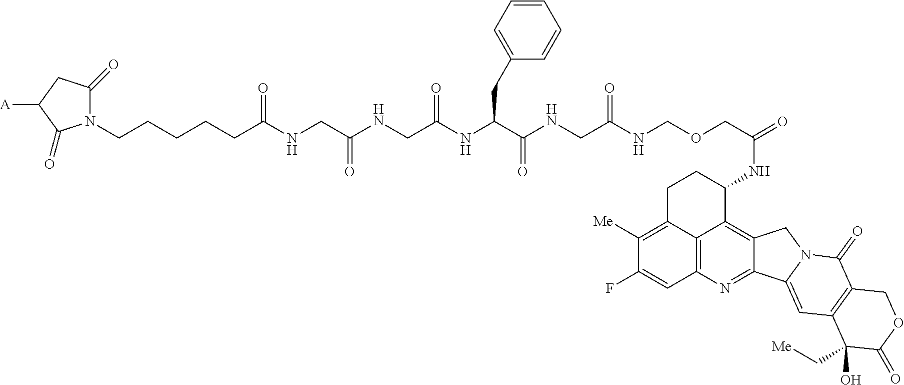
wherein A represents the connecting position to the antibody,
wherein the drug-linker is conjugated to the anti-HER2 antibody via a thioether bond; and
wherein the average number of units of the drug-linker conjugated per antibody molecule in the antibody-drug conjugate is in the range of from 3.5 to 4.5.

112. The method according to claim 95, wherein the antibody specific for a tumor antigen is an anti-GPR20 antibody.

113. The method according to claim 112, wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

114. The method according to claim 113, wherein the anti-GPR20 antibody lacks a lysine residue at the carboxyl terminus of the heavy chain.

115. The method according to claim 112, wherein the anti-GPR20 antibody is a part of an anti-GPR20 antibody-drug conjugate further comprising a linker and a drug, wherein the anti-GPR20 antibody is connected to the drug via the linker, the linker and the drug forming a drug-linker, wherein the drug-linker is as represented by the following formula: 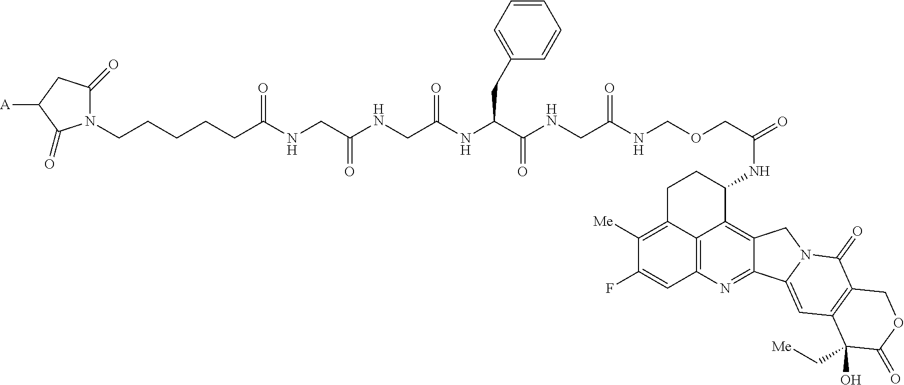
wherein A represents the connecting position to the antibody,
wherein the drug-linker is conjugated to the anti-GPR20 antibody via a thioether bond; and
wherein the average number of units of the drug-linker conjugated per antibody molecule in the antibody-drug conjugate is in the range of from 7 to 8.

116. The method according to claim 95, wherein the antibody specific for a tumor antigen is an anti-CDH6 antibody.

117. The method according to claim 116, wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

118. The method according to claim 117, wherein the anti-CDH6 antibody lacks a lysine residue at the carboxyl terminus of the heavy chain.

119. The method according to claim 116 wherein the anti-CDH6 antibody is a part of an anti-CDH6 antibody-drug conjugate further comprising a linker and a drug, wherein the anti-CDH6 antibody is connected to the drug via the linker, the linker and the drug forming a drug-linker, wherein the drug-linker is as represented by the following formula: 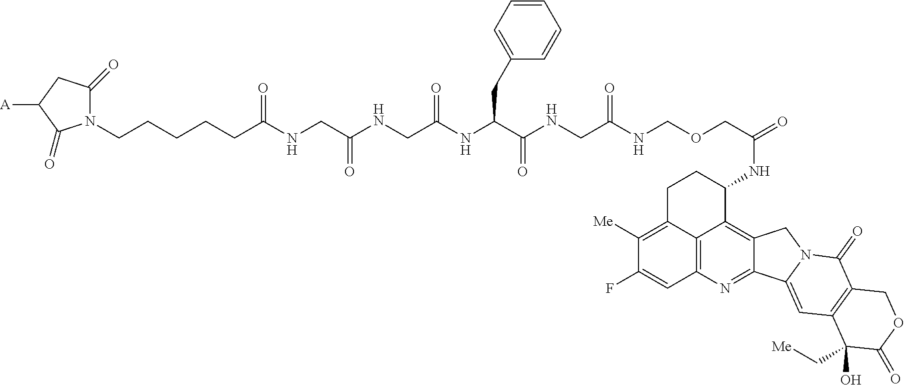
wherein A represents the connecting position to the antibody,
wherein the drug-linker is conjugated to the anti-CDH6 antibody via a thioether bond; and
wherein the average number of units of the drug-linker conjugated per antibody molecule in the antibody-drug conjugate is in the range of from 7 to 8.

120. The method according to claim 81, wherein the CD47 inhibitor and the antibody specific for a tumor antigen are separately contained active components in different formulations.

121. The method according to claim 81, wherein the method is for treating at least one selected from the group consisting of: breast cancer, gastric cancer, colorectal cancer, lung cancer, esophageal cancer, salivary gland cancer, esophagogastric junction adenocarcinoma, biliary tract cancer, Paget's disease, pancreatic cancer, ovarian cancer, bladder cancer, prostate cancer, uterine carcinosarcoma, head and neck cancer, hepatocellular cancer, cervical cancer, brain tumor, glioma, eye tumor, thyroid cancer, thymus cancer, gallbladder cancer, lymphoma, leukemia, and myelodysplastic syndrome.


SEQUENCE LISTING (16 sequences)

SEQ ID NO: 1 - protein, 450 aa, synthetic construct
  REGION 1..450: note = Heavy chain of anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
  REGION 1..214: note = Light chain of anti-HER2 antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, synthetic construct
  REGION 1..447: note = Heavy chain of anti-HER3 antibody
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 220 aa, synthetic construct
  REGION 1..220: note = Light chain of anti-HER3 antibody
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 470 aa, synthetic construct
  REGION 1..470: note = Heavy chain of anti-TROP2 antibody
MKHLWFFLLLVAAPRWVLSQVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 234 aa, synthetic construct
  REGION 1..234: note = Light chain of anti-TROP2 antibody
MVLQTQVFISLLLWISGAYGDIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 471 aa, synthetic construct
  REGION 1..471: note = Heavy chain of anti-B7-H3 antibody
MKHLWFFLLLVAAPRWVLSQVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 233 aa, synthetic construct
  REGION 1..233: note = Light chain of anti-B7-H3 antibody
MVLQTQVFISLLLWISGAYGEIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 472 aa, synthetic construct
  REGION 1..472: note = Heavy chain of anti-GPR20 antibody
MKHLWFFLLLVAAPRWVLSEVQLVQSGAEVKKPGASVKVSCKASGYTFTSYYISWIRQAPGQGLKYMGFINPGSGHTNYNEKFKGRVTITADKSSSTATMELSSLRSEDTAVYYCARGAGGFLRIITKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 234 aa, synthetic construct
  REGION 1..234: note = Light chain of anti-GPR20 antibody
MVLQTQVFISLLLWISGAYGDTQLTQSPSSLSASVGDRVTITCRASKSVSTYIHWYQQKPGKQPKLLIYSAGNLESGVPSRFSGSGSGTDFTLTISSLQPEDFANYYCQQINELPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 471 aa, synthetic construct
  REGION 1..471: note = Heavy chain of anti-CDH6 antibody
MKHLWFFLLLVAAPRWVLSEVQLVQSGAEVKKPGASVKVSCKASGYTFTRNFMHWVRQAPGQGLEWMGWIYPGDGETEYAQKFQGRVTITADTSTSTAYMELSSLRSEDTAVYYCARGVYGGFAGGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 233 aa, synthetic construct
  REGION 1..233: note = Light chain of anti-CDH6 antibody
MVLQTQVFISLLLWISGAYGDIQMTQSPSSLSASVGDRVTITCKASQNIYKNLAWYQQKPGKAPKLLIYDANTLQTGVPSRFSGSGSGSDFTLTISSLQPEDFATYFCQQYYSGWAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 14 - protein, 504 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 15 - protein, 376 aa, synthetic construct
  REGION 1..376: note = Sequence of ALX148_hIgG1
MEPAGPAPGRLGPLLCLLLAASCAWSGVAGEEELQIIQPDKSVLVAAGETATLRCTITSLFPVGPIQWFRGAGPGRELIYNQREGPFPRVTTVSDTTKRNNMDFSIRIGAITPADAGTYYCVKFRKGSPDDVEFKSGAGTELSVRAKPSDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 371 aa, synthetic construct
  REGION 1..371: note = Sequence of ALX148_mIgG1
MEPAGPAPGRLGPLLCLLLAASCAWSGVAGEEELQIIQPDKSVLVAAGETATLRCTITSLFPVGPIQWFRGAGPGRELIYNQREGPFPRVTTVSDTTKRNNMDFSIRIGAITPADAGTYYCVKFRKGSPDDVEFKSGAGTELSVRAKPSGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK